(19) United States
(12) Patent Application Publication
(10) Pub. No.: US 2023/0272341 A1
(43) Pub. Date: Aug. 31, 2023

(54) MULTIFUNCTIONAL IMMUNE EFFECTOR CELL AND USE THEREOF

(71) Applicant: CRAGE MEDICAL CO., LIMITED, Hong Kong (CN)

(72) Inventors: Zonghai LI, Shanghai (CN); Ruixin SUN, Shanghai (CN)

(73) Assignee: CRAGE MEDICAL CO., LIMITED, Hong Kong (CN)

(21) Appl. No.: 18/020,488

(22) PCT Filed: Aug. 10, 2021
(86) PCT No.: PCT/CN2021/111838

§ 371 Date: May 10, 2023

(30) Foreign Application Priority Data
Aug. 10, 2020 (CN) 202010795298.5

(51) Int. Cl. C12N 5/0783; C07K 16/28; C07K 16/40; A61P 35/00; C12N 15/62

(52) U.S. Cl. CPC C12N 5/0636; C07K 16/28; C07K 16/40; A61P 35/00; C12N 15/62; C07K 2317/565; C07K 2317/24; C07K 2319/03

(57) ABSTRACT

Provided is an immune effector cell targeting FAP and another tumor-associated antigen, which can improve a tumor microenvironment, kill tumor cells, and can be used to treat tumors.

This application claims priority of Chinese patent application CN202010795298.5 filed on Aug. 10, 2020, which is hereby incorporated by reference in its entirety.

### TECHNICAL FIELD

The present application relates to the field of tumor immunotherapy, more particularly, to immune effector cells targeting FAP and another tumor-associated antigen and applications thereof.

### BACKGROUND ART

Tumors, especially solid tumors, are complexes composed of tumor cells and their surrounding stromal cells and non-cellular components. The occurrence and development of tumors is a dynamic process of mutual promotion and co-evolution between tumor cells and their microenvironment. The tumor microenvironment plays an important role in growth and metastasis of a tumor. Cancer associated fibroblasts (CAFs), as one of the most important components in the tumor microenvironment, are characterized by expression of α-smooth muscle actin (α-SMA) and fibroblast activating protein (FAP); it can secrete a variety of cytokines to promote tumor angiogenesis, induce epithelial-mesenchymal transition of tumor cells, break the homeostasis between tissue cells, and make the microenvironment more conducive to tumor growth. CAFs cells have a promoting effect on many common cancers, such as breast cancer, liver cancer, gastric cancer, colorectal cancer, ovarian cancer, lung cancer, and pancreatic cancer. In recent years, treatment of cancer by targeting CAFs cells has gradually become a new idea. FAP is specifically expressed in CAFs cells, thus the effect of killing CAFs cells can be achieved by targeting FAP.

Fibroblast activating protein (FAP) is an antigen molecule expressed on CAFs cells (NCBI reference number: NP_001278736.1). At present, it has been reported that PT-100, a small molecule dipeptidyl peptidase inhibitor, targets FAP to inhibit CAFs; in a breast cancer model, pirfenidone (PFD) (as an anti-fibrotic drug targeting CAFs) together with doxorubicin can effectively inhibit tumor growth and lung metastasis.

### SUMMARY OF THE APPLICATION

The object of the present application is to provide a multifunctional immune effector cell to improve the killing effect of the immune effector cell on tumor cells such as pancreatic cancer.

In order to achieve the above object, the technical solutions provided by the application are as follows:

In a first aspect, the present application provides a multifunctional immune effector cell, wherein the immune effector cell expresses a protein specifically recognizing FAP and a protein specifically recognizing a tumor-associated antigen.

In a particular embodiment, the tumor-associated antigen is a solid tumor-associated antigen; preferably, the solid tumor-associated antigen is an antigen associated with breast cancer, liver cancer, gastric cancer, colorectal cancer, ovarian cancer, lung cancer, or pancreatic cancer; more preferably, the solid tumor is pancreatic cancer; or the solid tumor-associated antigen is Claudin 18.2.

In a particular embodiment, the cell is selected from the group consisting of: T cell, NK cell, NKT cell, macrophage, CIK cell, and stem cell-derived immune effector cell; preferably, the cell is T cell.

In a particular embodiment, the protein specifically recognizing FAP and the protein specifically recognizing a tumor-associated antigen are expressed into a fusion protein by fused expression; preferably, the fusion protein is connected with a transmembrane domain and an intracellular signal domain to form a chimeric receptor.

In a particular embodiment, the chimeric receptor comprises a protein specifically recognizing FAP, a protein specifically recognizing a tumor-associated antigen, a transmembrane domain and an intracellular signal domain which are connected in sequence; alternatively, the chimeric receptor comprises a protein specifically recognizing a tumor-associated antigen, a protein specifically recognizing FAP, a transmembrane domain and an intracellular signal domain which are connected in sequence.

In a particular embodiment, the protein specifically recognizing FAP comprises an antibody targeting FAP or a ligand of FAP; preferably, the antibody targeting FAP is a single chain antibody or a single domain antibody; more preferably, the single chain antibody has LCDR1, LCDR2 and LCDR3 represented by SEQ ID NOs: 35, 36 and 37, and HCDR1, HCDR2 and HCDR3 represented by SEQ ID NOs: 38, 39 and 40; more preferably, the single chain antibody has the amino acid sequence represented by SEQ ID NO: 2.

In a particular embodiment, the protein specifically recognizing a tumor-associated antigen is an antibody specifically recognizing a tumor antigen or a ligand of a tumor antigen; preferably, the antibody specifically recognizing a tumor antigen is a single chain antibody or a single domain antibody; more preferably, the single chain antibody has LCDR1, LCDR2 and LCDR3 represented by SEQ ID NOs: 29, 30 and 31, and HCDR1, HCDR2 and HCDR3 represented by SEQ ID NOs: 26, 27 and 28; more preferably, the single chain antibody has the amino acid sequence represented by SEQ ID NO: 4.

In a particular embodiment, the chimeric receptor is selected from the group consisting of: chimeric antigen receptor (CAR), chimeric T cell receptor, or T cell antigen coupler (TAC).

In a particular embodiment, the protein specifically recognizing FAP and the protein specifically recognizing a tumor-associated antigen are connected through a connecting peptide, preferably the protein specifically recognizing a tumor-associated antigen is located upstream of the protein specifically recognizing FAP.

In a particular embodiment, the intracellular signal domain is selected from the intracellular signal domain sequences of CD3ζ, FcεRIγ, CD27, CD28, CD137 and CD134, or a combination thereof.

In a particular embodiment, the chimeric receptor comprises an extracellular binding domain, a transmembrane domain and an intracellular signal domain which are connected in the following order:
a fusion protein, a transmembrane domain of CD8, and an intracellular domain of CD3ζ;
a fusion protein, a transmembrane domain of CD8, an intracellular signal domain of CD137, and an intracellular domain of CD3ζ;
a fusion protein, a transmembrane domain of CD28, an intracellular signal domain of CD28, and an intracellular domain of CD3ζ; or
a fusion protein, a transmembrane domain of CD28, an intracellular signal domain of CD28, an intracellular signal domain of CD137, and intracellular domain of CD3ζ.

In a particular embodiment, the protein specifically recognizing FAP and the protein specifically recognizing a tumor-associated antigen are expressed separately.

In a particular embodiment, the protein specifically recognizing FAP is a chimeric receptor which comprises an antibody targeting FAP or a ligand of FAP, a transmembrane domain, and an intracellular signal domain.

In a particular embodiment, the protein specifically recognizing a tumor-associated antigen is a chimeric receptor which comprises an antibody targeted-binding a tumor antigen or a ligand of a tumor antigen, a transmembrane domain, and an intracellular signal domain.

In a particular embodiment, the protein specifically recognizing FAP is a chimeric receptor A which comprises an antibody targeting FAP or a ligand of FAP, a transmembrane domain and an intracellular signal domain; and the protein specifically recognizing a tumor-associated antigen is a chimeric receptor B which comprises an antibody targeted-binding a tumor antigen or a ligand of a tumor antigen, a transmembrane domain, and an intracellular signal domain.

In a particular embodiment, the chimeric receptor A and the chimeric receptor B have the same intracellular signal domain or different intracellular signal domains.

In a particular embodiment, the intracellular signal domain is selected from the intracellular signal domain sequences of CD3ζ, FcεRIγ, CD27, CD28, CD137 and CD134, or a combination thereof; preferably, the chimeric receptor A has the amino acid sequence represented by SEQ ID NO: 43, 44, 45 or 46; or the chimeric receptor B has the amino acid sequence represented by SEQ ID NO: 16, 32, 33 or 34.

In a particular embodiment, the chimeric receptor has the amino acid sequence represented by SEQ ID NO: 41, SEQ ID NO: 20, SEQ ID NO: 22 or SEQ ID NO: 42; preferably, the chimeric receptor has the amino acid sequence represented by SEQ ID NO: 41 or 42.

In a second aspect, the present application provides a fusion protein which comprises a protein targeting FAP, a protein targeted-specifically recognizing FAP, and a protein specifically recognizing a tumor-associated antigen.

In a particular embodiment, the tumor-associated antigen is a solid tumor-associated antigen; preferably, the solid tumor-associated antigen is an antigen associated with breast cancer, liver cancer, gastric cancer, colorectal cancer, ovarian cancer, lung cancer, or pancreatic cancer; more preferably, the solid tumor-associated antigen is Claudin 18.2.

In a particular embodiment, the fusion protein is connected with a transmembrane domain and an intracellular signal domain to form a chimeric receptor.

In a particular embodiment, the chimeric receptor comprises a protein specifically recognizing FAP, a protein specifically recognizing a tumor-associated antigen, a transmembrane domain and an intracellular signal domain which are connected in sequence; alternatively, the chimeric receptor comprises a protein specifically recognizing a tumor-associated antigen, a protein specifically recognizing FAP, a transmembrane domain and an intracellular signal domain which are connected in sequence.

In a particular embodiment, the protein specifically recognizing FAP comprises an antibody targeting FAP or a ligand of FAP; preferably, the antibody targeting FAP is a single chain antibody or a single domain antibody; more preferably, the single chain antibody has the amino acid sequence represented by SEQ ID NO: 2.

In a particular embodiment, the protein specifically recognizing a tumor-associated antigen is an antibody specifically recognizing a tumor antigen or a ligand of a tumor antigen; preferably, the antibody specifically recognizing a tumor antigen is a single chain antibody or a single domain antibody; more preferably, the single chain antibody has the amino acid sequence represented by SEQ ID NO: 4.

In a particular embodiment, the chimeric receptor is selected from the group consisting of: chimeric antigen receptor (CAR), chimeric T cell receptor, or T cell antigen coupler (TAC).

In a particular embodiment, the protein specifically recognizing FAP and the protein specifically recognizing a tumor-associated antigen are connected through a connecting peptide, preferably the protein specifically recognizing a tumor-associated antigen is located upstream of the protein specifically recognizing FAP.

In a particular embodiment, the intracellular signal domain is selected from the intracellular signal domain sequences of CD3ζ, FcεRIγ, CD27, CD28, CD137 and CD134, or a combination thereof.

In a particular embodiment, the chimeric receptor comprises an extracellular binding domain, a transmembrane domain and an intracellular signal domain which are connected in the following order:
a fusion protein, a transmembrane domain of CD8, and an intracellular domain of CD3ζ;
a fusion protein, a transmembrane domain of CD8, an intracellular signal domain of CD137, and an intracellular domain of CD3ζ;
a fusion protein, a transmembrane domain of CD28, an intracellular signal domain of CD28, and an intracellular domain of CD3ζ; or
a fusion protein, a transmembrane domain of CD28, an intracellular signal domain of CD28, an intracellular signal domain of CD137, and intracellular domain of CD3ζ.

In a third aspect, the present application provides a nucleic acid encoding any one of the fusion proteins according to the second aspect of the present application.

In a fourth aspect, the present application provides an expression vector comprising the nucleic acid according to the third aspect of the present application.

In a fifth aspect, the present application provides a virus comprising the nucleic acid according to the third aspect of the present application or comprising the expression vector according to the fourth aspect of the present application.

In a sixth aspect, the present application provides a pharmaceutical composition which comprises: any one of the immune effector cells according to the first aspect of the present application, or any one of the fusion proteins according to the second aspect of the present application; and a pharmaceutically acceptable carrier.

In a seventh aspect, the present application provides a kit which comprises the pharmaceutical composition according to the sixth aspect of the present application; or any one of the immune effector cells according to the first aspect of the present application; or any one of the fusion proteins according to the second aspect of the present application.

In a eighth aspect, the present application provides a method for treating a tumor, which comprises administering any one of the immune effector cells according to the first aspect of the present application to an individual suffering from a tumor, preferably the lymphocytes of the individual are eliminated before administration of the immune effector cells.

In a particular embodiment, the tumor is a tumor rich in a large number of CAFs cells in the tumor microenvironment; preferably, the tumor is breast cancer, liver cancer, gastric cancer, lung cancer, or pancreatic cancer; more preferably, the tumor is pancreatic cancer.

### Beneficial Effects of the Present Application

The construction of dual-target immune effector cells modified by chimeric antigen receptor aims to kill tumor cells on the one hand and CAFs cells on the other hand, thereby improving the tumor microenvironment for the better treatment of a tumor.

### BRIEF DESCRIPTION OF THE DRAWINGS

FIG. 1A shows the MSCV-CLDN18.2-BBZ plasmid map, FIG. 1B shows the MSCV-FAP-BBZ plasmid map, FIG. 1C shows the MSCV-FAP/CLDN18.2-BBZ plasmid map, and FIG. 1D shows the MSCV-CLDN18. 2/FAP-BBZ plasmid map;

FIG. 2 shows the positive rate of CAR-T cell infection;

FIG. 3 shows the cytotoxicity of CLDN18.2-BBZ CAR T cells, FAP-BBZ CAR T cells, CLDN18.2/FAP-BBZ CART cells and FAP/CLDN18.2-BBZ CART cells on tumor cells in vitro;

FIG. 4 shows the in vivo efficacy of CAR-T cells on the mouse subcutaneous xenograft tumor model bearing PANC02-A2 pancreatic cancer cells: FIG. 4A shows the growth curve of xenograft tumor volume, FIG. 4B shows the measurement results of mouse body weight, FIG. 4C shows the measurement results of xenograft tumor weight, and FIG. 4D shows the tumor inhibition rate of CLDN18.2-BBZ, FAP-BBZ, CLDN18.2-FAP-BBZ, FAP-CLDN18.2-BBZ CAR-T cells on the treatment of PANC02-A2 pancreatic cancer cell xenograft tumors.

### DETAILED DESCRIPTION OF THE APPLICATION

After in-depth research, the present inventors first revealed an immune effector cell modified with chimeric antigen receptor which can simultaneously recognizes FAP and another tumor-associated antigen, and the immune effector cell can be used to treat a tumor rich in a large number of CAFs cells in the tumor microenvironment.

Unless defined otherwise, all technical and scientific terms used herein have the same meaning as commonly understood by one of skill in the fields of gene therapy, biochemistry, genetics and molecular biology. Methods and materials similar or equivalent to those described herein can be used in the practice or testing of the present application. All publications, patent applications, patents, and other references mentioned herein are hereby incorporated by reference in their entirety. In case of conflict, the present specification, including definitions, shall prevail. In addition, the materials, methods, and examples are illustrative only and not intended to be limiting unless otherwise specified.

Unless otherwise indicated, the practice of the present application employs conventional techniques of cell biology, cell culture, molecular biology, transgenic biology, microbiology, recombinant DNA and immunology, which are within the skill of the art. These techniques are fully described in the literatures, for example, Current Protocols in Molecular Biology (Frederick M. AUSUBEL, 2000, Wiley and son Inc., Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higginseds. 1984); B. D. Hames & S. J. Higginseds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes I-IV (D. M. Weir and C. C. Blackwell, eds., 1986); and Manipulating the Mouse Embryo (Cold Spring Harbor Laboratory Press, Cold Spring Harbor, N.Y., 1986).

In order to better understand the present application, relevant terms are defined as follows:

The term “single domain antibody” (sdAb), also called nanobody, consists of a single antibody variable domain. A single domain antibody has small molecular weight and strong stability; although it has a simple structure, it can still achieve a binding affinity to a specific antigen that is comparable to or even higher than that of a traditional antibody. Therefore, single domain antibodies are widely used in bispecific antibodies, as well as cell therapy (such as chimeric antigen receptor T cells).

The term “chimeric receptor” refers to a fusion molecule formed by linking DNA fragments from different sources or corresponding cDNAs of proteins by genetic recombination technology, comprising an extracellular domain, a transmembrane domain and an intracellular domain. Chimeric receptors include, but are not limited to: chimeric antigen receptor (CAR), chimeric T cell receptor (TCR), T cell antigen coupler (TAC).

The term “T cell receptor (TCR)” mediates T cell recognition of specific major histocompatibility complex (MHC)-restricted peptide antigen, including classical TCR receptors and optimized TCR receptors. A classical TCR receptor consists of two peptide chains (a and (3), and each peptide chain can be divided into a variable region (V region), a constant region (C region), a transmembrane region and a cytoplasmic region, etc., and its antigen specificity exists in the V region, and the V region (Vα, or Vβ) has three hypervariable regions (CDR1, CDR2, and CDR3). In one aspect, for T cells expressing classical TCR, the specificity of the TCR of the T cells to a target antigen can be induced by using, for example, antigen stimulation to the T cells.

The term “T cell antigen coupler (TAC)” comprises three functional domains: 1. an antigen binding domain, including single chain antibody, designed ankyrin repeat protein (DARPin), or other targeting groups; 2. an extracellular region domain, a single chain antibody that binds to CD3ζ, so that the TAC receptor and the TCR receptor are close; 3. an transmembrane region and an intracellular region of the CD4 co-receptor, wherein the intracellular region is linked to protein kinase LCK, catalyzes the phosphorylation of immunoreceptor tyrosine-based activation motifs (ITAMs) of the TCR complex as an initial step in T cell activation.

The term “chimeric T cell receptor” includes recombinant polypeptides derived from various polypeptides constituting the TCR, which can bind to surface antigens on target cells, and interact with other polypeptides of the complete TCR complex, and are usually co-localized at T cell surface. A chimeric T cell receptor consists of a TCR subunit and an antigen-binding domain composed of a human or humanized antibody domain, wherein the TCR subunit comprises at least part of the TCR extracellular domain, transmembrane domain, the stimulation domain of the intracellular signal domain of the TCR intracellular domain; the TCR subunit is operably linked to the antibody domain, wherein the extracellular, transmembrane, and intracellular signal domain of the TCR subunit are derived from CD3ε or CD3γ, and the chimeric T cell receptor is integrated into the TCR expressed on T cells.

The term “chimeric antigen receptor” (CAR) comprises an extracellular antigen binding domain, a transmembrane domain and an intracellular signaling domain. The intracellular signaling domain comprises a functional signaling domain of a stimulatory molecule and/or a co-stimulatory molecule; in one aspect, the stimulatory molecule is a ζ chain bound to a T cell receptor complex; in one aspect, a cytoplasmic signaling domain further comprises functional signaling domains of one or more co-stimulatory molecules, such as 4-1BB (i.e., CD137), CD27 and/or CD28.

The term “extracellular binding domain” comprises an antibody or a ligand that specifically recognizes an antigen (such as a tumor antigen), and preferably the antibody is a single chain antibody or a single domain antibody. More preferably, the extracellular antigen-binding region of the chimeric antigen receptor is connected to the transmembrane domain of CD8 or CD28 through the hinge region of CD8, and the transmembrane domain is followed by the intracellular signal domain. In this solution, the extracellular binding domain comprises 1 or 2 antibodies, preferably, an antibody targeting FAP and/or an antibody targeting another tumor-associated antigen, and the two antibodies can be connected through a connecting peptide.

The term “transmembrane domain” refers to a region of a protein sequence that spans a cell membrane, and it may comprise one or more additional amino acids adjacent to the transmembrane domain, for example, one or more amino acids associated with the extracellular region of the protein from which the transmembrane protein is derived (e.g., 1, 2, 3, 4, 5, 6, 7, 8, 9, 10 up to 15 amino acids of the extracellular region), and/or one or more additional amino acids associated with the intracellular region of the protein from which the transmembrane domain is derived (e.g., 1, 2, 3, 4, 5, 6, 7, 8, 9, 10 up to 15 amino acids of the intracellular region). In one aspect, the transmembrane domain is a domain that is related to one of the other domains of the chimeric receptor; for example, in one embodiment, the transmembrane domain may be from the same protein which the signaling domain, co-stimulatory domain, or the hinge domain is derived from. In some cases, a transmembrane domain may be selected, or modified by amino acid substitution to avoid binding of such a domain to a transmembrane domain of the same or different surface membrane protein, for example, to minimize the interaction with other members of the receptor complex. In one aspect, a transmembrane domain is capable of homo-dimerizing with another chimeric receptor on the surface of a cell expressing chimeric receptors. The transmembrane domain may be derived from a natural or recombinant source. When the source is natural, the domain may be derived from any membrane-bound protein or transmembrane protein. In one aspect, the transmembrane domain is capable of signaling to the intracellular domain whenever the chimeric receptor is bound to a target. Transmembrane domains particularly used in the present application may include at least the following transmembrane domains: for example, the α, β or ζ chain of a T-cell receptor, CD28, CD27, CD3ε, CD45, CD4, CD5, CD8, CD9, CD16, CD22, CD33, CD37, CD64, CD80, CD86, CD134, CD137, and CD154. In some embodiments, the transmembrane domain may include at least the following transmembrane domains: e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, and IPO-3), BLAME (SLAMF8), SELPLG (CD162), LTBR, PAG/Cbp, NKG2D, and NKG2C.

In certain instances, a transmembrane domain may be linked to the extracellular region of the CAR (i.e., the antigen binding domain of the CAR) by a hinge (e.g., a hinge from a human protein). Optionally, a short oligopeptide or polypeptide linker in a length of 2-10 amino acids may form a bond between the transmembrane domain and the cytoplasmic region of the CAR. The glycine-serine duplex provides a particularly suitable linker.

The term “signaling domain” refers to a functional portion of a protein that functions by transmitting information within a cell, so as to regulate the cell activity via a definite signaling pathway by producing a second messenger or by acting as an effector in response to such a messenger. An intracellular signaling domain may comprise the entire intracellular portion of the molecule, or the entire natural intracellular signaling domain, or a functional fragment or derivative thereof.

The term “co-stimulatory molecule” refers to a signal that binds to a cell-stimulating signal molecule (e.g., TCR/CD3), and such a combination causes T cell proliferation, and/or up-regulation or down-regulation of key molecules.

The terms “activation” and “excitation” are used interchangeably, and may refer to a process by which a cell transforms from a quiescent state to an active state. The process can include responses to phenotypic or genetic changes in antigen, migration, and/or functional activity status. For example, the term “activation” may refer to a process by which T cells are gradually activated. For example, T cells may require at least one signal to be fully activated.

The term “intracellular signal domain” comprises an intracellular signaling domain. The intracellular signaling domain refers to a part of the protein that transduces immune effector function signals and guides cells to perform specific functions, and it can guide the activation of immune effector function of immune cells. The immune effector function of T cells can be, for example, cytolytic activity or helper activity, including secretion of cytokines. While the entire intracellular signaling domain can generally be used, in many cases it is not necessary to use the entire chain, and a truncated portion can be used instead of the full chain, as long as the immune effector function signal is transduced.

The “intracellular signal domain” may also comprise a co-stimulatory signal domain, which can enhance the proliferation ability of immune cells and the secretion function of cytokines by activating the intracellular signaling domain of immune effector cells, thereby prolonging the survival time of immune cells.

The term “tumor-associated antigen” refers to an antigen expressed in a tumor. The “tumor-associated antigen” can be selected from (but not limited to): EGFR, GPC3, HER2, EphA2, Claudin18.1, Claudin18.2, Claudin 6, GD2, EpCAM, mesothelin, CD19, CD20, ASGPR1, EGFRvIII, de4EGFR, CD19, CD33, IL13R, LMP1, PLAC 1, NY-ESO-1, MAGE4, MUC1, MUC16, LeY, CEA, CAIX (carbonic anhydrase IX), CD123.

The term “solid tumor” refers to a tangible tumor. A tangible mass that can be found by clinical examination such as X-ray film, CT scan, B-ultrasound, or palpation is usually called solid tumor. “Solid tumor” can also mean that although a tangible mass is not found by clinical examination such as X-ray film, CT scan, B-ultrasound, or palpation, the subject shows the expression of antigens of solid tumor.

In the present application, various tumors known in the art can be comprised in the present application, as long as the tumor expresses (or highly expresses) CAFs.

As used herein, “GPC3” or “glypican 3” is a member of the glypican family, which plays an important role in regulation of cell growth and differentiation. Abnormal expression of GPC3 is closely related to the occurrence and development of various tumors, such as abnormal expression in liver cancer, lung cancer, breast cancer, ovarian cancer, kidney cancer, thyroid cancer, gastric cancer, colorectal cancer, etc.

In the present application, immune effector cells target GPC3-positive tumors. In a particular embodiment, the tumors include but are not limited to: liver cancer, gastric cancer, lung cancer, esophageal cancer, head and neck cancer, bladder cancer, ovarian cancer, cervical cancer, kidney cancer, pancreatic cancer, cervical cancer, liposarcoma, melanoma, adrenal gland cancer, schwannoma, malignant fibrous histiocytoma, esophageal cancer; preferably liver cancer, gastric cancer, lung cancer, and esophageal cancer.

The term “claudin 18.2” or “claudin 18A2” (CLD18.2, CLD18A2, CLDN18A2, or CLDN18.2) herein may also refer to a homologue, ortholog, interspecies homologue, codon-optimized form, truncated form, fragmented form, mutated form or any other known derived form (e.g., a post-translationally modified variant) of the known claudin 18A2 sequence. In some embodiments, the claudin 18A2 is a peptide having GenBank accession number NP_001002026 (mRNA: NM 001002026), having the sequence represented by SEQ ID NO: 23.

The term “CAFs”, also known as tumor-associated fibroblasts, are the most abundant host cells in the microenvironment of solid tumors, and acquire an activated phenotype under the influence of the microenvironment. Different from normal fibroblasts, CAFs are characterized by the expression of α-smooth muscle actin (α-SMA) and fibroblast activation protein (FAP), and they can secrete a large number of growth factors (such as VEGF, TGF-β, hepatocyte growth factor, etc.), and can synthesize and deposit ECM, produce various collagens and cohesin, and mediate ECM remodeling. The importance of CAFs in the process of tumor occurrence and development, metastasis and recurrence has been verified, and it has been revealed that they promote tumor growth by dominating the tumor microenvironment.

The term “FAP” is also called fibroblast activation protein, which belongs to the class of serine proteases, and is a dimer consisting of two subunits, i.e., FAPα (a molecular weight of 95 kDa) and FAPβ (a molecular weight of 105 kDa), with a molecular weight of 170 kDa. FAP can be selectively expressed on more than 90% of activated fibroblasts in lung, breast and colorectal cancer stroma. FAPα has the sequence represented by SEQ ID NO: 24.

The term “antibody” refers to a protein or polypeptide sequence derived from an immunoglobulin molecule that specifically binds an antigen. An antibody can be polyclonal or monoclonal, multi-chain or single-chain, a whole immunoglobulin, or antibody fragment; and can be derived from a natural or recombinant source. An antibody can be a tetramer of immunoglobulin molecules.

Herein “single chain antibody (scFv)” refers to an antibody as defined by the following, which is a recombinant protein comprising a heavy chain variable region (VH) and a light chain variable region (VL) connected by a linker; and these two domains are brought into association by the linker to ultimately form an antigen binding site. Preferably, a single chain antibody is a sequence of one amino acid chain encoded by one nucleotide chain. The single chain antibody used in the present application can be further modified by conventional techniques known in the art alone or in combination, e.g., amino acid deletion, insertion, substitution, addition, and/or recombination, and/or other modification methods. Methods for introducing such modifications into the DNA sequence of an antibody based on its amino acid sequence are well known to those skilled in the art; for example, Sambrook, Molecular Cloning: A Laboratory Manual, Cold Spring Harbor Laboratory (2002) N.Y. The modifications referred to are preferably carried out at the nucleic acid level. The above single chain antibody may also include the derivatives thereof.

The immune effector cells modified by chimeric antigen receptor according to the present application can be applied to the preparation of pharmaceutical compositions or diagnostic reagents. In addition to the effective amount of the immune cells, the composition may also comprise a pharmaceutically acceptable carrier. The term “pharmaceutically acceptable” means that the molecular entities and compositions do not produce adverse, allergic or other adverse reactions when they are properly administered to animals or humans, for example, cell cryoprotectants. The term “cell cryoprotectant” may be a composition, for example, may comprise isotonic saline, buffer saline, glycerol, DMSO, ethylene glycol, propylene glycol, acetamide, polyvinylpyrrolidone (PVP), sucrose, poly ethylene glycol, dextran, albumin and hydroxyethyl starch, serum, etc.

The composition of the present application can be made into various dosage forms according to needs, and can be administered by a physician according to the patient's type, age, body weight and general disease condition, administration method and other factors to determine a dosage beneficial to the patient. The administration method can be injection or other therapeutic methods.

The term “lymphocyte depletion” or “lymphocyte clearance” refers to the depletion of lymphocytes in a subject. It includes administration of a lymphocyte depleting agent, whole body radiation therapy, or a combination thereof. For example, in order to increase the expansion or later maintenance of immune effector cells in a subject, before, at the same time, after, or any combination of administrating therapeutically effective amount of CAR-T cells for therapy, one or more agents capable of substantially depleting the subject's lymphocytes, whole body radiation therapy, or a combination thereof can be administered to the subject alone or in combination.

The lymphocyte depleting agent can be an antineoplastic chemotherapeutic agent, for example, fludarabine, cyclophosphamide, or a combination thereof. A physician can choose a specific lymphocyte depleting agent and the appropriate dose according to the subject to be treated, e.g., CAMPATH, anti-CD3 antibody, cyclosporine, FK506, rapamycin, mycophenolic acid, steroid, FR901228, melphalan, cyclophosphamide, fludarabine, and whole body radiation therapy.

The immune effector cells are administrated before, during, and after the lymphocyte depletion therapy, and they can also be administered in combination, i.e., administrating before and during, before and after, during and after, or before, during and after the lymphocyte depletion therapy. In some embodiments, the lymphocyte depletion therapy is performed 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month prior to the immune effector cell therapy, or any combination thereof. In some embodiments, the lymphocyte depletion therapy is performed 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month after the immune effector cell therapy, or any combination thereof.

The multifunctional immune effector cell provided in this application expresses a protein specifically recognizing FAP, and a protein specifically recognizing a tumor-associated antigen.

In a particular embodiment, the protein specifically recognizing FAP comprises an antibody targeting FAP or a ligand of FAP, and the antibody targeting FAP is a full-length antibody or an antibody fragment. The antibody fragment refers to an antibody that comprises binding ability of a full-length antibody but only has a partial structure of a full-length antibody. Examples of an antibody fragment include but are not limited to: Fv, Fab, Fab′, Fab′-SH, F(ab′)₂, single chain antibody (scFv), single domain antibody, bispecific antibody, and multi-specific antibody formed from antibody fragments.

In a particular embodiment, the protein specifically recognizing claudin18.2 comprises an antibody targeting FAP or a ligand of FAP, and the antibody targeting claudin18.2 is a full-length antibody or an antibody fragment thereof. The antibody fragment refers to an antibody that comprises binding ability of a full-length antibody but only has a partial structure of the full-length antibody. Examples of the antibody fragment include but are not limited to: Fv, Fab, Fab′, Fab′-SH, F(ab′)2, single chain antibody (scFv), single domain antibody, bispecific antibody, and multi-specific antibody formed from antibody fragments.

In a particular embodiment, the protein specifically recognizing FAP is connected to the protein specifically recognizing claudin18.2 to form a fusion protein. For example, the scFv of the protein specifically recognizing FAP is connected to the scFv of the protein specifically recognizing claudin18.2 to form a fusion protein. The protein recognizing FAP can be directly connected to the protein specifically recognizing claudin18.2, or they can be connected through a linker, for example, through one to five G4S connecting peptides. Alternatively, in another particular embodiment, a protein comprising an antibody specifically recognizing FAP is connected to a protein comprising an antibody specifically recognizing claudin18.2 to form a fusion protein, for example, a chimeric receptor comprising an antibody specifically recognizing FAP is connected to a chimeric receptor comprising an antibody specifically recognizing claudin18.2 to form a fusion protein. In a particular embodiment, the fusion protein can also be connected to the transmembrane and intracellular domains to form a chimeric protein; for example, the chimeric protein comprises a fusion protein, a transmembrane domain, and an intracellular signal domain which are connected in sequence. In a particular embodiment, the chimeric protein may have the sequence represented by SEQ ID NO: 41 or 42, or the sequence represented by SEQ ID NO: 20 or 22. In the sequence represented by SEQ ID NO: 20, 22, 41 or 42, the intracellular signal domain and the transmembrane domain can be replaced according to techniques known to those skilled in the art, for example, replacing by other transmembrane domain or intracellular signal domain. Therefore, in some embodiments, the chimeric protein can comprise the protein of the sequence represented by the extracellular region of SEQ ID NO: 41 or 42; for example, the chimeric protein comprises the sequence of positions 1-506 in SEQ ID NO: 41 or 42.

In a particular embodiment, the protein specifically recognizing FAP and the protein specifically recognizing claudin18.2 are expressed separately. For example, a chimeric receptor comprising an antibody specifically recognizing FAP and a chimeric receptor comprising an antibody specifically recognizing claudin18.2 are expressed on immune effector cells, respectively. For example, the protein specifically recognizing FAP is a chimeric receptor A that comprises an antibody targeting FAP or a ligand of FAP, a transmembrane domain, and an intracellular signal domain; the protein specifically recognizing and binding a tumor-associated antigen is a chimeric receptor B that comprises an antibody targeted-binding to a tumor antigen or a ligand of the tumor antigen, a transmembrane domain and an intracellular signal domain; wherein the chimeric receptor A and the chimeric receptor B are respectively expressed. In a particular embodiment, the chimeric receptor A and the chimeric receptor B have the same intracellular signal domain or different intracellular signal domains. In a particular embodiment, the intracellular signal domain is selected from the intracellular signal domain sequences of CD3ζ, FcεRIγ, CD27, CD28, CD137 and CD134, or a combination thereof. In practice, these sequences are preferably of human origin. In a particular embodiment, the chimeric receptor A has the amino acid sequence represented by SEQ ID NO: 43, 44, 45, or 46. In some embodiments, the chimeric receptor A may also have the sequence represented by SEQ ID NO: 18. In a particular embodiment, the chimeric receptor B has the amino acid sequence represented by SEQ ID NO: 16, 32, 33, or 34. In some embodiments, the chimeric receptor B may also have the amino acid sequence encoded by the nucleic acid sequence represented by SEQ ID NO: 15.

In the present application, the tumor-associated antigen is a solid tumor-associated antigen; preferably, the solid tumor-associated antigen is an antigen associated with breast cancer, liver cancer, gastric cancer, colorectal cancer, ovarian cancer, lung cancer, and pancreatic cancer. In a particular embodiment, said solid tumor is pancreatic cancer. In another particular embodiment, the solid tumor-associated antigen is Claudin 18.2.

In the present application, the term “immune effector cells” has the same meaning as “immune cells”, and refers to cells that participate in the immune response and produce immune effects, such as T cells, B cells, natural killer (NK) cells, natural killer T (NKT) cells, dendritic cells, CIK cells, macrophages, mast cells, etc., and they can also be artificially engineered cells with the function of immune effector cells.

In some embodiments, the immune effector cells are T cells, NK cells, NKT cells, macrophages, CIK cells, and stem cell-derived immune effector cells. In some embodiments, the T cells may be autologous T cells, allogeneic T cells, or allogeneic T cells. In some embodiments, the NK cells may be allogeneic NK cells.

The term “artificially engineered cell with immune effector cell function” refers to a cell or cell line without immune effector acquires immune effector cell function after being artificially engineered or stimulated by a stimulant. For example, 293T cells are artificially engineered to have the function of immune effector cells; for example, stem cells are induced in vitro to differentiate into immune effector cells.

In some instances, “T cells” may be pluripotent stem cells derived from bone marrow that differentiate and mature into immunocompetent mature T cells within the thymus. In some cases, “T cells” may be a population of cells with specific phenotypic characteristics, or a mixed population of cells with different phenotypic characteristics; for example, “T cells” may be cells comprising at least one subset of T cells: stem cell-like memory T cells (Tscm cells), central memory T cells (Tcm), effector T cells (Tef, Teff), regulatory T cells (tregs) and/or effector memory T cells (Tem). In some cases, “T cells” may be a specific subtype of T cells, such as γδT cells.

T cells can be obtained from many sources, including PBMC, bone marrow, lymph node tissue, cord blood, thymus tissue, and tissues from infection sites, ascites, pleural effusion, spleen tissues and tumors. In some cases, T cells can be obtained from blood collected from an individual by using any number of techniques known to those of skill in the art, e.g., Ficoll™ isolation. In one embodiment, the cells from the circulating blood of the individual are obtained by apheresis. Apheresis products usually comprise lymphocytes, including T cells, monocytes, granulocytes, B cells, other nucleated leucocytes, red blood cells, and platelets. In one embodiment, the cells collected by apheresis can be washed to remove plasma molecules, then placing the cells in a suitable buffer or culture medium for subsequent processing steps. Alternatively, the cells can be derived from a healthy donor, or from a patient diagnosed with cancer.

The present application will be further described in combination with particular examples. It should be understood that, these examples are only used to illustrate the present application, and are not intended to limit the scope of the present application. The experimental methods that do not indicate specific conditions in the following examples, are usually performed according to conventional conditions e.g., J. Sambrook et al., eds., Molecular Cloning: A Laboratory Manual (Third Edition), Science Press, 2002, or as recommended by the manufacturer.

### Example 1: Construction of Immune Effector Cells Modified by Chimeric Antigen Receptor

1. Construction of MSCV-Claudin18.2-BBZ, MSCV-FAP-BBZ, MSCV-FAP-Claudin18.2-BBZ, MSCV-Claudin18.2-FAP-BBZ Plasmids

In this example, conventional molecular biology methods in the field and the following materials were used: the scFv targeting FAP, wherein the nucleotide sequence is represented by SEQ ID NO: 1, and the amino acid sequence is represented by SEQ ID NO: 2; the scFv targeting Claudin 18.2, wherein the nucleotide sequence is represented by SEQ ID NO: 3, and the amino acid sequence is represented by SEQ ID NO: 4; and a second-generation chimeric antigen receptor, which has a transmembrane domain of CD8, an intracellular domain of 4-1BB (CD137), and an intracellular domain of CD3ζ.

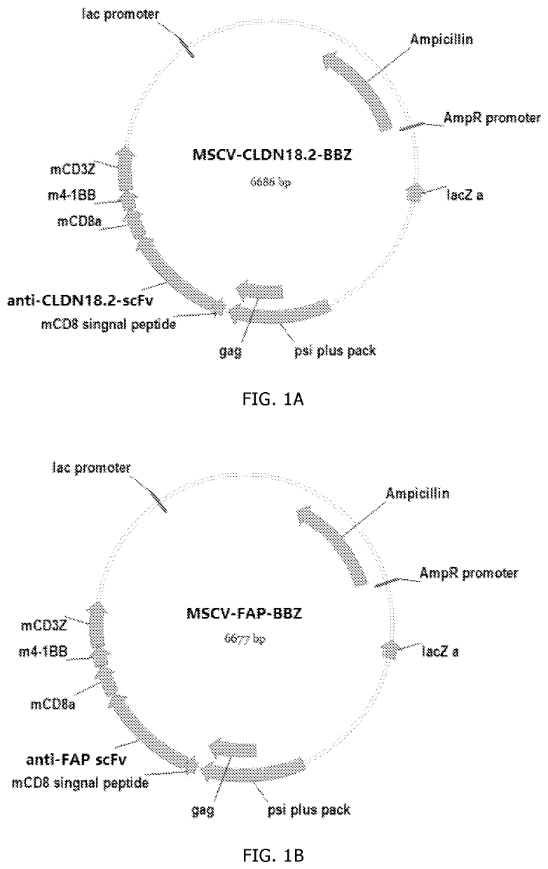

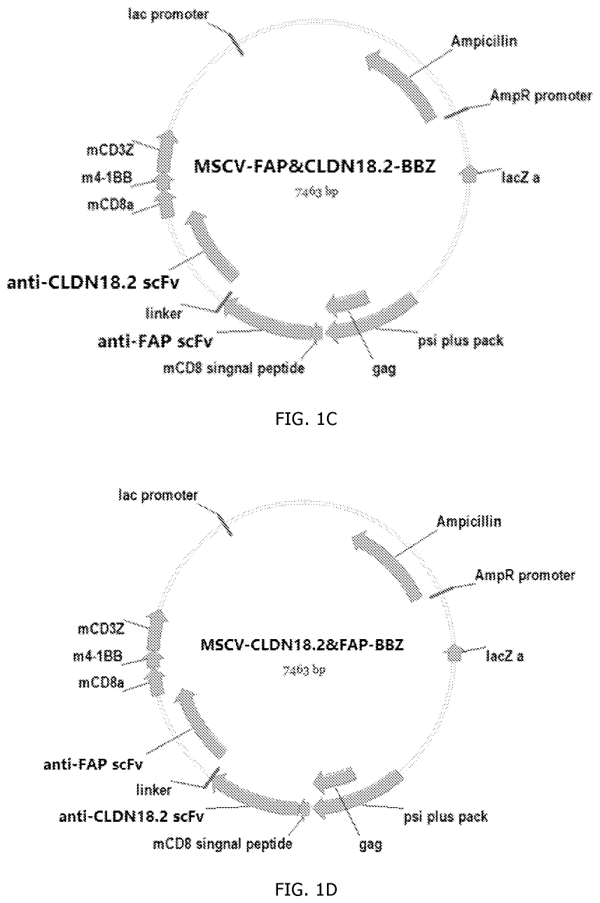

Referring to the plasmid map shown in FIG. 1, plasmids MSCV-CLDN18.2-BBZ (FIG. 1A), MSCV-FAP-BBZ (FIG. 1B), MSCV-FAP-CLDN18.2-BBZ (FIG. 1C), and MSCV-CLDN18.2-FAP-BBZ (FIG. 1D) were constructed respectively.

MSCV-IRES-GFP (purchased from Addgene) was used as a vector to construct the retroviral plasmids MSCV-CLDN18.2-BBZ, MSCV-FAP-BBZ, MSCV-FAP-CLDN18.2-BBZ and MSCV-CLDN18.2-FAP-BBZ which express the second-generation chimeric antigen receptors.

The CLDN18.2-BBZ sequence comprises the mouse CD8a signal peptide (the nucleotide sequence is represented by SEQ ID NO: 5, and the amino acid sequence is represented by SEQ ID NO: 6), the scFv targeting Claudin 18.2 (the nucleotide sequence is represented by SEQ ID NO: ID NO: 3, and the amino acid sequence is represented by SEQ ID NO: 4), mouse CD8 hinge region and transmembrane domain (the nucleotide sequence is represented by SEQ ID NO: 7, and the amino acid sequence is represented by SEQ ID NO: 8), mouse 4-1BB intracellular signaling domain (the nucleotide sequence is represented by SEQ ID NO: 9, and the amino acid sequence is represented by SEQ ID NO: 10), and mouse CD3 intracellular domain (the nucleotide sequence is represented by SEQ ID NO: 11, and the amino acid sequence is represented by SEQ ID NO: 12).

The FAP-BBZ sequence comprises the mouse CD8a signal peptide (the nucleotide sequence is represented by SEQ ID NO: 5, and the amino acid sequence is represented by SEQ ID NO: 6), the scFv targeting FAP (the nucleotide sequence is represented by SEQ ID NO: 1, and the amino acid sequence is represented by SEQ ID NO: 2), mouse CD8 hinge region and transmembrane domain (the nucleotide sequence is represented by SEQ ID NO: 7, and the amino acid sequence is represented by SEQ ID NO: 8), mouse 4-1BB intracellular signaling domain (the nucleotide sequence is represented by SEQ ID NO: 9, and the amino acid sequence is represented by SEQ ID NO: 10), and mouse CD3 intracellular domain (the nucleotide sequence is represented by SEQ ID NO: 11, and the amino acid sequence is represented by SEQ ID NO: 12).

The FAP-CLDN18.2-BBZ sequence consists of: the mouse CD8a signal peptide (the nucleotide sequence is represented by SEQ ID NO: 5, and the amino acid sequence is represented by SEQ ID NO: 6), the scFv targeting FAP (the nucleotide sequence is represented by SEQ ID NO: 1, and the amino acid sequence is represented by SEQ ID NO: 2), the connecting peptide (G45)3 (the nucleotide sequence is represented by SEQ ID NO: 13, and the amino acid sequence is represented by SEQ ID NO: 14), the scFv targeting Claudin 18.2 (the nucleotide sequence is represented by SEQ ID NO: 3, and the amino acid sequence is represented by SEQ ID NO: 4), the mouse CD8 hinge region and transmembrane domain (the nucleotide sequence is represented by SEQ ID NO: 7, and the amino acid sequence is represented by SEQ ID NO: 8), the mouse 4-1BB intracellular signaling domain (the nucleotide sequence is represented by SEQ ID NO: 9, and the amino acid sequence is represented by SEQ ID NO: 10), and intracellular fragment CD3 of mouse CD3 (the nucleotide sequence is represented by SEQ ID NO: 11, and the amino acid sequence is represented by SEQ ID NO: 12).
2. The plasmids of MSCV-CLDN18.2-BBZ, MSCV-FAP-BBZ, MSCV-FAP-CLDN18.2-BBZ, and MSCV-CLDN18.2-FAP-BBZ were respectively transfected into 293T for packaging retroviruses to obtain retroviruses.
3. T cell activation: lymphocytes were obtained by grinding the spleen of C57BL/6 mice, after being treated with CD3+ mouse T cell negative screening kit, the obtained mouse CD3+ T lymphocytes were added into Dynabeads Mouse T-activator CD3/CD28 magnetic beads at a volume ratio of 1:1 for activation and stimulation, then putting into cell culture incubator, wherein the medium is RPMI 1640 complete medium (10% FBS+50 μM β-mercaptoethanol+100U/mL IL-2+1 ng/mL IL-7).

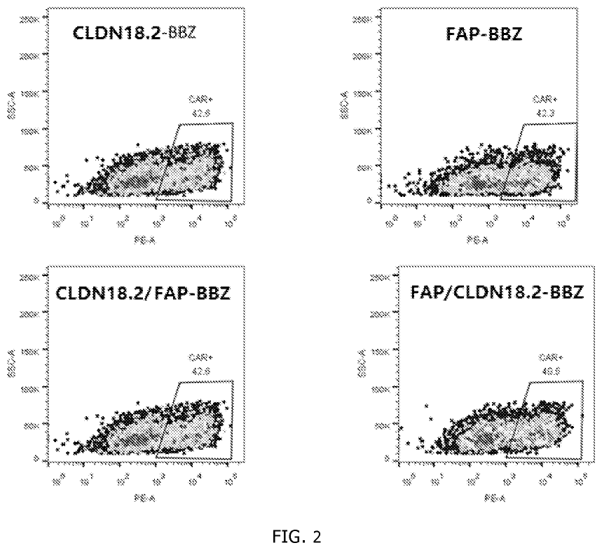

Mouse spleen CD3+ T lymphocytes activated for 24 hours were inoculated in a 24-well plate coated with Retronectin (5 μg/mL), adding retrovirus to infect for 24 hours, then replacing with fresh medium to obtain mouse CLDN18.2-BBZ CART cells, FAP-BBz CART cells, CLDN18.2-FAP-BBZ CART cells, and FAP-CLDN18.2-BBZ CART cells. The positive rates of the infection of the above CAR-T cells are shown in FIG. 2. It can be seen from FIG. 2 that, the positive rate of CLDN18.2-BBZ cell infection is 42.6%, the positive rate of FAP-BBZ cell infection is 42.3%, the positive rate of CLDN18.2-FAP-BBZ cell infection is 42.6%, and the positive rate of FAP-CLDN18.2-BBZ cell infection is 40.5%.

### Example 2: In Vitro Killing Toxicity Experiment of CAR-T on Mouse Pancreatic Cell PANC02-A2

2.1 Construction of Mouse Pancreatic Cancer Cell PANC02-A2 Expressing Claudin18.2

The full-length sequence of mouse-derived CLDN18.2 was overexpressed by using a lentiviral vector in the mouse pancreatic cancer cell line PANC02 (purchased from ATCC) cells, to obtain a stably expressed claudin18.2-positive PANC02-A2 cell line. The PANC02-A2 cell line was screened by flow cytometry sorting technology, and this cell line was used to carry out the follow-up studies. PANC02 cells were used as negative control cells for the follow-up experiments.

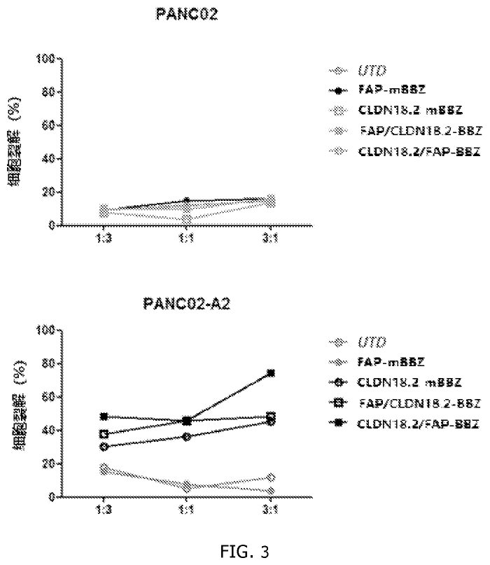

2.2 The untreated mouse T cells (UTD), and CLDN18.2-BBZ CAR T cells (the nucleotide sequence of CLDN18.2-BBZ is represented by SEQ ID NO: 15, and the amino acid sequence is represented by SEQ ID NO: 16), FAP-BBz CAR T cells (the nucleotide sequence of FAP-BBz is represented by SEQ ID NO: 17, and the amino acid sequence is represented by SEQ ID NO: 18), CLDN18.2-FAP-BBZ CAR T cells (the nucleotide sequence of CLDN18.2-FAP-BBZ is represented by SEQ ID NO: 19, and the amino acid sequence is represented by SEQ ID NO: 20), and FAP-CLDN18.2-BBZ CART cells (the nucleotide sequence of FAP-CLDN18.2-BBZ is represented by SEQ ID NO: 21, and the amino acid sequence is represented by SEQ ID NO: 22) in Example 1 were taken to co-incubate with PANC02 cells, PANC02-A2 cells respectively at the ratio of 1:3, 1:1, and 3:1, after co-incubating for 16 h, the secretion of LDH in the supernatant was detected by using Cytox 96 Non-Radioactive Cytotoxicity Assay, then calculating killing toxicity (as shown in FIG. 3) of the following cells on tumor cells: the UTD, CLDN18.2-BBZ CAR T cells (represented by CLDN18.2 mBBZ in FIG. 3), FAP-BBz CAR T cells (represented by FAP mBBZ in FIG. 3), CLDN18.2-FAP-BBZ CAR T cells (represented by CLDN18.2/FAP BBZ in FIG. 3), and FAP-CLDN18.2-BBZ CAR T cells (represented by FAP/CLDN18.2BBZ in FIG. 3). For specific detection steps and calculation methods, see the instructions of Promaga Cytox 96 Non-Radioactive Cytotoxicity Assay (Promaga Company, REF: G1782).

It can be seen from FIG. 3 that, FAP-BBZ CAR T cells have a weaker tumor killing effect, which is comparable to that of UTD. Both FAP-CLDN18.2-BBZ CAR T cells and CLDN18.2-FAP-BBZ CART cells show relatively good tumor cell killing effect.

### Example 3: Anti-Tumor Efficacy of CAR-T Cells on Subcutaneously Xenograft Tumors of Mouse Pancreatic Cancer

(1) Establishment and Grouping of Subcutaneous Xenograft Tumor Model of Mouse Pancreatic Cancer:

Well-growing PANC02-A2 cells in the logarithmic growth phase were collected, and 1×10⁶ cells were subcutaneously inoculated into C57BL/6 mice (mice with normal immune system), and the day of tumor cell inoculation was recorded as Day 0.
(2) On the 10th day after tumor inoculation (i.e., Day 10), mice were administered cyclophosphamide by intraperitoneal injection. Dosage of cyclophosphamide: 100 mg/kg. 0.2 g of cyclophosphamide powder was fully dissolved in 20 ml of normal saline, and 200 μl was injected intraperitoneally into each mouse.
(3) On the 11th day after tumor inoculation (i.e., Day 11), CART cells (2×10⁶) were injected by tail vein. CLDN18.2-BBZ, FAP-BBz, CLDN18.2/FAP-BBZ and FAP/CLDN18.2-BBZ cells were constructed as described in Step 1 of Example 1 of the application.

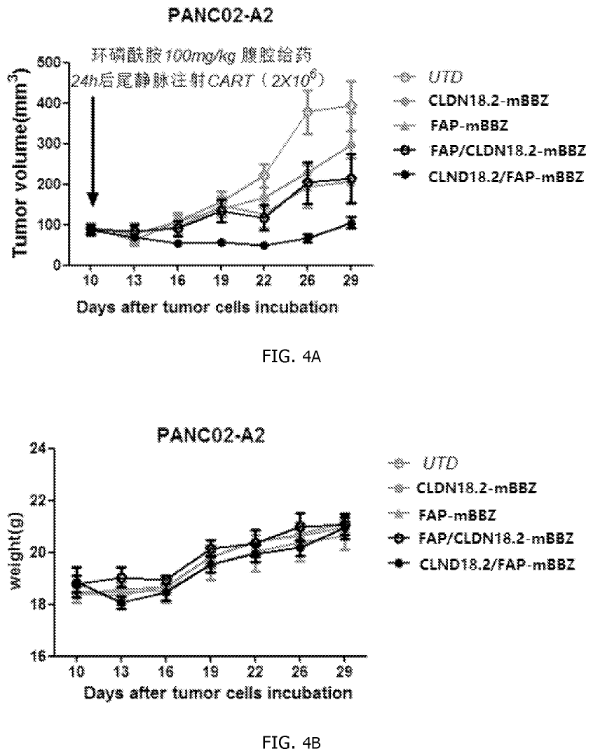

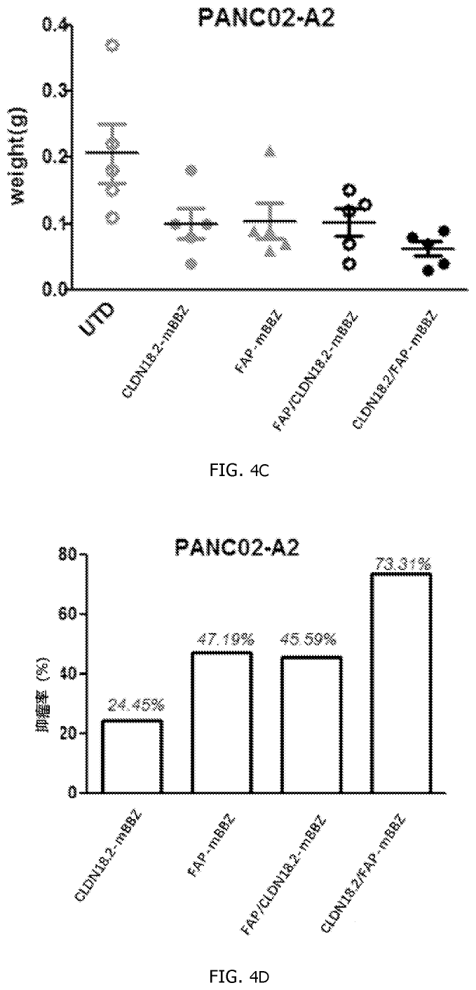

The mice were divided into 5 groups, 5 mice in each group:
UTD group: 2×10⁶ mouse T cells without virus transduction were administered;
CLDN18.2-BBZ group (represented by CLADN18.2-mBBZ in FIG. 4): 2×10⁶ CLDN18.2-BBZ-CAR-T cells were administered;
FAP-BBZ group (represented by FAP mBBZ in FIG. 4): 2×10⁶ FAP-BBZ-CAR-T cells were administered;
FAP/CLDN18.2-BBZ group: 2×10⁶ FAP-CLDN18.2-BBZ-CAR-T cells were administered;
CLDN18.2/FAP-BBZ group: 2×10⁶ CLDN18.2-FAP-BBZ-CAR-T cells were administered;
(4) Detection of tumor volume. The changes in the tumor volume of the mice were observed and measured continuously to record three times a week. The formula for calculating tumor volume is: tumor volume=(tumor length*tumor width²)/2.

The detection results of tumor volume in mice are shown in FIG. 4A, and the results show that CAR-T cells in the CLDN18.2/FAP-BBZ group can significantly inhibit the tumor volume in mice. At the same time, it was detected that the body weight of mice in each group do not change significantly (as shown in FIG. 4B), suggesting that the dual-target and single-target CART do not cause obvious toxic effects on the mice.
(5) Measurement of tumor weight. On Day 29, the mice were euthanized, the tumors of the mice were removed to weigh the tumor weights, and the specific statistical results are shown in FIG. 4C. It is suggested that the CAR-T cells in the CLDN18.2/FAP-BBZ group have a better anti-tumor effect on pancreatic cancer in mice.
(6) Calculation of the tumor inhibition rate. The final tumor volume values on Day 29 of the mice were used for calculation, and the calculation formula is: tumor inhibition rate (%)=[(final tumor volume value of mice in UTD group—final tumor volume value of mice in experimental group)/final tumor volume value of mice in UTD group]*100. As shown in FIG. 4D, the tumor inhibition rate in the CLDN18.2-BBZ group is 24.45%, and it does not achieve a good effect on tumor growth inhibition; while the tumor inhibition rate in the FAP-BBZ group is 47.19%, the tumor inhibition rate in the FAP/CLDN18.2-BBZ group is 45.59%, and the tumor inhibition rate in the CLDN18.2/FAP-BBZ group is 73.31%.

### Example 4: Effect of Dual-Target CAR-T on the Microenvironment of Mouse Pancreatic Cancer

The mice of each group treated with CAR-T cells in Example 3 were taken to separate the tumor tissues on Day 21 for flow cytometry analysis, and the MDSC cells, Treg cells, Macrophage cells and DC cells were detected respectively. The detection results show that, the dual-target CLDN18.2/FAP-BBZ group can inhibit the infiltration of MDSC cells.

Exemplarily, the antibodies used in the above examples are represented by SEQ ID NO: 2 and 4, but it should be understood that the antibodies used herein can be mouse antibodies or humanized, and the transmembrane domain and intracellular domain used herein can also derived from different species (e.g., human) according to different purposes.

Exemplarily, although CAR-T cells were used in the above examples, the T cells can also express other cytokines that enhance the function of CAR-T cells, such as CAR-T cells co-expressing CAR and type I interferon, and CAR-T cells co-expressing CAR and PD1, etc.

Exemplarily, although CAR-T cells were used in the above examples, other immune cells (such as NK cells and NK-T cells) can also be selected, and specific subtypes of immune cells (such as γ/δT cells) can also be selected.

Exemplarily, CARs of mouse origin were selected in the above examples, but its signal peptide, hinge region, transmembrane region, etc. can be selected from other species according to different purposes, including but not limited to: human signal peptide, hinge region, transmembrane region, and intracellular region; for example, according to different purposes, the antibody can also be selected from mouse antibody, humanized antibody, or complete human antibody against different targets, the sequence of a fusion protein used herein can be the sequence represented by SEQ ID NO: 41 or 42.

All documents mentioned in this application are incorporated herein by reference as if each is individually incorporated by reference. In addition, it should be understood that after reading the above teaching content of the present application, those skilled in the art can make various changes or modifications to the present application, and these equivalent forms also fall within the scope defined by the claims of the present application.

The sequence used herein is as follows:

| SEQ | | |
|---|---|---|
| ID | Sequence | |
| NO: | names | Sequences |
| 1 | Nucleic acid | CAGGTGCAATTGGTGCAGTCTGGGGCTGAGGTGAAGAAGCCTGGGTCCTCGGTGAAGGTCTCCTGCAAGG |
| | sequence of | CCTCCGGAGGCACATTCAGCAGCTACGCTATAAGCTGGGTGCGACAGGCCCCTGGACAAGGGCTCGAGTGG |
| | anti-FAP | ATGGGAGGGATCATCCCTATCTTTGGTACAGCAAACTACGCACAGAAGTTCCAGGGCAGGGTCACCATTACT |
| | antibody | GCAGACAAATCCACGAGCACAGCCTACATGGAGCTGAGCAGCCTGAGATCTGAGGACACCGCCGTGTATTA |
| | | CTGTGCGAGAGATGCTGCTGATAGGGACTACTGGGGCCAAGGGACCACCGTGACCGTCTCCTCAGGTGGAG |
| | | GCGGTTCAGGCGGAGGTGGTTCTGGCGGTGGCGGATCGGATATTGTTATGACTCAATCTCCACTGTCTCTGC |
| | | CGGTGACTCCAGGCGAACCGGCGAGCATTTCTTGCCGTTCCAGCCAGTCTCTGCTTCACCCCAACGGCTTC |
| | | AACCATCTCTATTGGTACCTGCAAAAACCGGGTCAGAGCCCTCAGCTGCTGATCTACGTGGGGGGTAACCG |
| | | CGCTTCCGGTGTACCGGACCGTTTCAGCGGCTCTGGATCCGGCACCGATTTCACGTTGAAAATCAGCCGTGT |
| | | TGAAGCAGAAGACGTGGGCGTTTATTACTGTCAGCAGCGTAATAATAAGAATCGTACTTTTGGTCAAGGCAC |
| | | CAAGGTCGAAATTAAACGT |
| 2 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | anti-FAP | EPASISCRSSQSLLHPNGFNHLYWYLQKPGQSPQLLIYVGGNRASGVPDRFSGSGSGTDFTLKISRVEAEDVGVY |
| | antibody | YCQQRNNKNRTFGQGTKVEIKR |
| 3 | Nucleic acid | caggtgcagctgcaggagagcggccccggcctgatcaagcccagccagaccctgagcctgacctgcaccgtgagcggcggcagcatcagcagcggctacaactggcact |
| | sequence of | ggatccggcagccccccggcaagggcctggagtggatcggctacatccactacaccggcagcaccaactacaaccccgccctgcggagccgggtgaccatcagcgtgga |
| | anti-CLDN18. | caccagcaagaaccagttcagcctgaagctgagcagcgtgaccgccgccgacaccgccatctactactgcgcccggatctacaacggcaacagcttcccctactggggcca |
| | 2 antibody | gggcaccaccgtgaccgtgagcageggtggaggcggttcaggcggaggtggttctggcggtggcggatcggacatcgtgatgacccagagccccgacagcctggccgtg |
| | | agcctgggcgagegggccaccatcaactgcaagagcagccagagcctgttcaacagcggcaaccagaagaactacctgacctggtaccagcagaagcccggccagcccc |
| | | cgaggacgtggccgtgtactactgccagaacgcctacagcttcccctacaccttcggcggcggcaccaagctggagatcaagcgg |
| 4 | Amino acid | qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence of | vtvssggggggggsggggsdivmtqspdslavslgeratinckssqslfnsgnqknyltwyqqkpgqppklliywastresgvpdrfsgsgsgtdftltisslqaedvav |
| | anti-CLDN18. | yycqnaysfpytfgggtkleikr |
| | 2 antibody | |
| 5 | Nucleotide | atggcctcaccgttgacccgctttctgtcgctgaacctgctgctgctgggtgagtcgattatcctggggagtggagaagct |
| | sequence of | |
| | mouse CD8α | |
| | signal peptide | |
| 6 | Amino acid | MASPLTRFLSLNLLLLGESIILGSGEA |
| | sequence of | |
| | mouse CD8α | |
| | signal peptide | |
| 7 | Nucleotide | actactaccaagccagtgctgcgaactccctcacctgtgcaccctaccgggacatctcagccccagagaccagaagattgtcggccccgtggctcagtgaaggggaccggat |
| | sequence of | tggacttcgcctgtgatatttacatctgggcacccttggccggaatctgcgtggcccttctgctgtccttgatcatcactctcatctgctaccacaggagccga |
| | mouse CD8 | |
| | hinge region | |
| | and | |
| | transmembrane | |
| | domain | |
| 8 | Amino acid | TTTKPVLRTPSPVHPTGTSQPQRPEDCRPRGSVKGTGLDFACDIYIWAPLAGICVALLLSLIITLICYHRSR |
| | sequence of | |
| | mouse CD8 | |
| | hinge region | |
| | and | |
| | transmembrane | |
| | domain | |
| 9 | Nucleotide | aaatggatcaggaaaaaattcccccacatattcaagcaaccatttaagaagaccactggagcagctcaagaggaagatgcttgtagctgccgatgtccacaggaagaagaag |
| | sequence of | gaggaggaggaggctatgagctg |
| | mouse 4-1BB | |
| | intracellular | |
| | signaling | |
| | domain | |
| 10 | Amino acid | KWIRKKFPHIFKQPFKKTTGAAQEEDACSCRCPQEEEGGGGGYEL |
| | sequence of | |
| | mouse 4-1BB | |
| | intracellular | |
| | signaling | |
| | domain | |
| 11 | Nucleotide | agcaggagtgcagagactgctgccaacctgcaggaccccaaccagctctacaatgagctcaatctagggcgaagagaggaatatgacgtcttggagaagaagcgggctcg |
| | sequence of | ggatccagagatgggaggcaaacagcagaggaggaggaacccccaggaaggcgtatacaatgcactgcagaaagacaagatggcagaagcctacagtgagatcggcac |
| | mouse CD3 | aaaaggcgagaggcggagaggcaaggggcacgatggcctttaccagggtctcagcactgccaccaaggacacctatgatgccctgcatatgcagaccctggcc |
| 12 | Amino acid | SRSAETAANLQDPNQLYNELNLGRREEYDVLEKKRARDPEMGGKQQRRRNPQEGVYNALQKDKMAEAYSEIG |
| | sequence of | TKGERRRGKGHDGLYQGLSTATKDTYDALHMQTLA |
| | intracellular | |
| | fragment CD3ζ | |
| | of mouse CD3 | |
| 13 | Nucleotide | ggtggaggcggttcaggcggaggtggttctggcggtggcggatcg |
| | sequence of | |
| | (G₄S)₃ | |
| 14 | Amino acid | GGGGSGGGGSGGGGS |
| | sequence of | |
| | (G₄S)₃ | |
| 15 | Nucleotide | caggtgcagctgcaggagagcggccccggcctgatcaagcccagccagaccctgagcctgacctgcaccgtgagcggcggcagcatcagcagcggctacaactggcact |
| | sequence of | ggatccggcagccccccggcaagggcctggagtggatcggctacatccactacaccggcagcaccaactacaaccccgccctgcggagccgggtgaccatcagcgtgga |
| | CLDN18.2- | caccagcaagaaccagttcagcctgaagctgagcagcgtgaccgccgccgacaccgccatctactactgcgcccggatctacaacggcaacagcttcccctactggggcca |
| | mBBZ | gggcaccaccgtgaccgtgagcagcggtggaggcggttcaggcggaggtggttctggcggtggcggatcggacatcgtgatgacccagagccccgacagcctggccgtg |
| | | agcctgggcgagcgggccaccatcaactgcaagagcagccagagcctgttcaacagcggcaaccagaagaactacctgacctggtaccagcagaagcccggccagcccc |
| | | ccaagctgctgatctactgggccagcacccgggagagcggcgtgcccgaccggttcagcggcagcggcagcggcaccgacttcaccctgaccatcagcagcctgcaggc |
| | | cgaggacgtggccgtgtactactgccagaacgcctacagcttcccctacaccttcggggggcaccaagctggagatcaagcggactactaccaagccagtgctgcgaact |
| | | ccctcacctgtgcaccctaccgggacatctcagccccagagaccagaagattgtcggccccgtggctcagtgaaggggaccggattggacttcgcctgtgatatttacatctgg |
| | | gcacccttggccggaatctgcgtggcccttetgctgtccttgatcatcactctcatctgctaccacaggagccgaaaatggatcaggaaaaaattcccccacatattcaagcaacc |
| | | atttaagaagaccactggagcagetcaagaggaagatgcttgtagctgccgatgtccacaggaagaagaaggaggaggaggaggctatgagctgagcaggagtgcagaga |
| | | ctgctgccaacctgcaggaccccaaccagctctacaatgagctcaatctagggcgaagagaggaatatgacgtcttggagaagaagcgggctcgggatccagagatgggag |
| | | gcaaacagcagaggaggaggaacccccaggaaggegtatacaatgcactgcagaaagacaagatggcagaagcctacagtgagatcggcacaaaaggcgagaggcgg |
| | | agaggcaaggggcacgatggcctttaccagggtctcagcactgccaccaaggacacctatgatgccctgcatatgcagaccctggcc |
| 16 | Amino acid | Qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence-1 of | vtvssggggsggggggggsdivmtqspdslavslgeratinckssqslfnsgnqknyltwyqqkpgqppklliywastresgvpdrfsgsgsgtdftltisslqaedvav |
| | CLDN18.2- | yycqnaysfpytfgggtkleikrTttpaprpptpaptiasqplslrpeacrpaaggavhtrgldfacdIyiwaplagtcgvlllslvitlycKrgrkkllyifkqpfmrpvqttq |
| | BBZ | eedgcscrfpeeeeggcelrvkfsrsadapayqqgqnqlynelnlgrreeydvldkrrgrdpemggkpqrrknpqeglynelqkdkmaeayseigmkgerrrgkghd |
| | | glyqglstatkdtydalhmqalppr |
| 17 | Nucleotide | caggtgcaattggtgcagtctggggctgaggtgaagaagcctgggtcctcggtgaaggtctcctgcaaggcctccggaggcacattcagcagctacgctataagctgggtgc |
| | sequence of | gacaggcccctggacaagggctcgagtggatgggagggatcatccctatctttggtacagcaaactacgcacagaagttccagggcagggtcaccattactgcagacaaatc |
| | FAP-mBBz | cacgagcacagcctacatggagctgagcagcctgagatctgaggacaccgccgtgtattactgtgcgagagatgctgctgatagggactactggggccaagggaccaccgt |
| | | gaccgtctcctcaggtggaggcggttcaggcggaggtggttctggcggtggggatcggatattgttatgactcaatctccactgtctctgccggtgactccaggcgaaccggc |
| | | gagcatttettgccgttccagccagtctctgcttcaccccaacggcttcaaccatctctattggtacctgcaaaaaccgggtcagagccctcagctgctgatctacgtggggggta |
| | | accgcgcttccggtgtaccggaccgtttcagcggetctggatccggcaccgatttcacgttgaaaatcagccgtgttgaagcagaagacgtgggcgtttattactgtcagcagcg |
| | | taataataagaatcgtacttttggtcaaggcaccaaggtcgaaattaaacgtactactaccaagccagtgctgcgaactccctcacctgtgcaccctaccgggacatctcagccc |
| | | cagagaccagaagattgtcggccccgtggctcagtgaaggggaccggattggacttcgcctgtgatatttacatctgggcacccttggccggaatctgcgtggcccttctgctgt |
| | | ccttgatcatcactctcatctgctaccacaggagccgaaaatggatcaggaaaaaattcccccacatattcaagcaaccatttaagaagaccactggagcagctcaagaggaag |
| | | atgcttgtagctgccgatgtccacaggaagaagaaggaggaggaggaggctatgagctgagcaggagtgcagagactgctgccaacctgcaggaccccaaccagctctac |
| | | aatgagctcaatctagggcgaagagaggaatatgacgtcttggagaagaagcgggctcgggatccagagatgggaggcaaacagcagaggaggaggaacccccaggaa |
| | | ggcgtatacaatgcactgcagaaagacaagatggcagaagcctacagtgagatcggcacaaaaggcgagaggcggagaggcaaggggcacgatggcctttaccagggtc |
| | | tcagcactgccaccaaggacacctatgatgccctgcatatgcagaccctggcc |
| 18 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | FAP-mBBz | EPASISCRSSQSLLHPNGFNHLYWYLQKPGQSPQLLIYVGGNRASGVPDRFSGSGSGTDFTLKISRVEAEDVGVY |
| | | YCQQRNNKNRTFGQGTKVEIKRTTTKPVLRTPSPVHPTGTSQPQRPEDCRPRGSVKGTGLDFACDIYIWAPLAGI |
| | | CVALLLSLIITLICYHRSRKWIRKKFPHIFKQPFKKTTGAAQEEDACSCRCPQEEEGGGGGYELSRSAETAANLQ |
| | | DPNQLYNELNLGRREEYDVLEKKRARDPEMGGKQQRRRNPQEGVYNALQKDKMAEAYSEIGTKGERRRGKG |
| | | HDGLYQGLSTATKDTYDALHMQTLA |
| 19 | Nucleotide | caggtgcagctgcaggagagcggccccggcctgatcaagcccagccagaccctgagcctgacctgcaccgtgagcggcggcagcatcagcagcggctacaactggcact |
| | sequence of | ggatccggcagccccccggcaagggcctggagtggatcggctacatccactacaccggcagcaccaactacaaccccgccctgcggagccgggtgaccatcagcgtgga |
| | CLDN18.2-FA | caccagcaagaaccagttcagcctgaagctgagcagcgtgaccgccgccgacaccgccatctactactgcgcccggatctacaacggcaacagcttcccctactggggcca |
| | P-mBBZ | gggccaagggaccaccgtgaccgtctcctcaggtggaggcggttcaggcggaggtggttctggcggtggcggatcggatattgttatgactcaatctccactgtctctgccggt |
| | | agcctgggcgagcgggccaccatcaactgcaagagcagccagagcctgttcaacagcggcaaccagaagaactacctgacctggtaccagcagaagcccggccagcccc |
| | | ccaagctgctgatctactgggccagcacccgggagagcggcgtgcccgaccggttcagcggcagcggcagcggcaccgacttcaccctgaccatcagcagcctgcaggc |
| | | cgaggacgtggccgtgtactactgccagaacgcctacagcttcccctacaccttcggcggcggcaccaagctggagatcaagcggggtggaggcggttcaggcggaggtg |
| | | gttctggcggtggcggatcgcaggtgcaattggtgcagtctggggctgaggtgaagaagcctgggtcctcggtgaaggtctcctgcaaggcctccggaggcacattcagcag |
| | | ctacgctataagctgggtgcgacaggcccctggacaagggctcgagtggatgggagggatcatccctatctttggtacagcaaactacgcacagaagttccagggcagggtc |
| | | accattactgcagacaaatccacgagcacagcctacatggagctgagcagcctgagatctgaggacaccgccgtgtattactgtgcgagagatgctgctgatagggactactg |
| | | gggccaagggaccaccgtgaccgtctcctcaggtggaggcggttcaggcggaggtggttctggcggtggcggatcggatattgttatgactcaatctccactgtctctgccggt |
| | | gactccaggcgaaccggcgagcatttcttgccgttccagccagtctctgcttcaccccaacggcttcaaccatctctattggtacctgcaaaaaccgggtcagagccctcagctg |
| | | ctgatctacgtggggggtaaccgcgcttccggtgtaccggaccgtttcagcggctctggatccggcaccgatttcacgttgaaaatcagccgtgttgaagcagaagacgtggg |
| | | cgtttattactgtcagcagcgtaataataagaatcgtacttttggtcaaggcaccaaggtcgaaattaaacgtactactaccaagccagtgctgcgaactccctcacctgtgcaccc |
| | | taccgggacatctcagccccagagaccagaagattgtcggccccgtggctcagtgaggggaccggattggacttcgcctgtgatatttacatctgggcacccttggccggaa |
| | | tctgcgtggcccttctgctgtccttgatcatcactctcatctgctaccacaggagccgaaaatggatcaggaaaaaattcccccacatattcaagcaaccatttaagaagaccactg |
| | | gagcagctcaagaggaagatgcttgtagctgccgatgtccacaggaagaagaaggaggaggaggaggctatgagctgagcaggagtgcagagactgctgccaacctgca |
| | | ggaccccaaccagctctacaatgagctcaatctagggcgaagagaggaatatgacgtcttggagaagaagcgggctcgggatccagagatgggaggcaaacagcagagg |
| | | aggaggaacccccaggaaggcgtatacaatgcactgcagaaagacaagatggcagaagcctacagtgagatcggcacaaaaggcgagaggcggagaggcaagggg |
| | | cgatggcctttaccagggtctcagcactgccaccaaggacacctatgatgccctgcatatgcagaccctggcc |
| 20 | Amino acid | qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence of | vtvssggggggggsggggsdivmtqspdslavslgeratinckssqslfnsgnqknyltwyqqkpgqppklliywastresgvpdrfsgsgsgtdftltisslqaedvav |
| | CLDN18.2-FA | yycqnaysfpytfgggtkleikrGGGGSGGGGSGGGGSQVQLVQSGAEVKKPGSSVKVSCKASGGTFSSYAISWVRQAPG |
| | P3-mBBZ | QGLEWMGGIIPIFGTANYAQKFQGRVTITADKSTSTAYMELSSLRSEDTAVYYCARDAADRDYWGQGTTVTVSS |
| | | GGGGSGGGGSGGGGSDIVMTQSPLSLPVTPGEPASISCRSSQSLLHPNGFNHLYWYLQKPGQSPQLLIYVGGNR |
| | | ASGVPDRFSGSGSGTDFTLKISRVEAEDVGVYYCQQRNNKNRTFGQGTKVEIKRTTTKPVLRTPSPVHPTGTSQP |
| | | QRPEDCRPRGSVKGTGLDFACDIYIWAPLAGICVALLLSLIITLICYHRSRKWIRKKFPHIFKQPFKKTTGAAQEE |
| | | DACSCRCPQEEEGGGGGYELSRSAETAANLQDPNQLYNELNLGRREEYDVLEKKRARDPEMGGKQQRRRNPQ |
| | | EGVYNALQKDKMAEAYSEIGTKGERRRGKGHDGLYQGLSTATKDTYDALHMQTLA |
| 21 | Nucleotide | caggtgcaattggtgcagtctggggctgaggtgaagaagcctgggtcctcggtgaaggtctcctgcaaggcctccggaggcacattcagcagctacgctataagctgggtgc |
| | sequence of | gacaggcccctggacaagggctcgagtggatgggagggatcatccctatctttggtacagcaaactacgcacagaagttccagggcagggtcaccattactgcagacaaatc |
| | FAP-CLDN18. | cacgagcacagcctacatggagctgagcagcctgagatctgaggacaccgccgtgtattactgtgcgagagatgctgctgatagggactactggggccaagggaccaccgt |
| | 2-mBBZ | gaccgtctcctcaggtggaggcggttcaggcggaggtggttctggcggtggcggatcggatattgttatgactcaatctccactgtctctgccggtgactccaggcgaaccggc |
| | | gagcatttcttgccgttccagccagtctctgcttcaccccaacggcttcaaccatctctattggtacctgcaaaaaccgggtcagagccctcagctgctgatctacgtggggggta |
| | | accgcgcttccggtgtaccggaccgtttcagcggctctggatccggcaccgatttcacgttgaaaatcagccgtgttgaagcagaagacgtgggcgtttattactgtcagcagcg |
| | | taataataagaatcgtacttttggtcaaggcaccaaggtcgaaattaaacgtggtggaggcggttcaggcggaggtggttctggcggtggcggatcgcaggtgcagctgcagg |
| | | agagcggccccggcctgatcaagcccagccagaccctgagcctgacctgcaccgtgagcggcggcagcatcagcagcggctacaactggcactggatccggcagccccc |
| | | cggcaagggcctggagtggatcggctacatccactacaccggcagcaccaactacaaccccgccctgcggagccgggtgaccatcagcgtggacaccagcaagaaccag |
| | | ttcagcctgaagctgagcagcgtgaccgccgccgacaccgccatctactactgcgcccggatctacaacggcaacagcttcccctactggggccagggcaccaccgtgacc |
| | | gtgagcagcggtggaggcggttcaggcggaggtggttctggcggtggcggatcggacatcgtgatgacccagagccccgacagcctggccgtgagcctgggcgagcgg |
| | | gccaccatcaactgcaagagcagccagagcctgttcaacagcggcaaccagaagaactacctgacctggtaccagcagaagcccggccagccccccaagctgctgatcta |
| | | ctgggccagcacccgggagagcggcgtgcccgaccggttcagcggcagcggcagcggcaccgacttcaccctgaccatcagcagcctgcaggccgaggacgtggccgt |
| | | gtactactgccagaacgcctacagcttcccctacaccttcggcggcggcaccaagctggagatcaagcggactactaccaagccagtgctgcgaactccctcacctgtgcacc |
| | | ctaccgggacatctcagccccagagaccagaagattgtcggccccgtggctcagtgaaggggaccggattggacttcgcctgtgatatttacatctgggcacccttggccgga |
| | | atctgcgtggcccttctgctgtccttgatcatcactctcatctgctaccacaggagccgaaaatggatcaggaaaaaattcccccacatattcaagcaaccatttaagaagaccact |
| | | ggagcagctcaagaggaagatgcttgtagctgccgatgtccacaggaagaagaaggaggaggaggaggctatgagctgagcaggagtgcagagactgctgccaacctgc |
| | | aggaccccaaccagctctacaatgagctcaatctagggcgaagagaggaatatgacgtcttggagaagaagcgggctcgggatccagagatgggaggcaaacagcagag |
| | | gaggaggaacccccaggaaggcgtatacaatgcactgcagaaagacaagatggcagaagcctacagtgagatcggcacaaaaggcgagaggcggagaggcaaggggc |
| | | acgatggcctttaccagggtctcagcactgccaccaaggacacctatgatgccctgcatatgcagaccctggcc |
| 22 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | FAP-CLDN18. | EPASISCRSSQSLLHPNGFNHLYWYLQKPGQSPQLLIYVGGNRASGVPDRFSGSGSGTDFTLKISRVEAEDVGVY |
| | 2-mBBZ | YCQQRNNKNRTFGQGTKVEIKRGGGGSGGGGSGGGGSqvqlqesgpglikpsqtlsltctvsggsissgynwhwirqppgkglewigyih |
| | | ytgstnynpalrsrvtisvdtsknqfslklssvtaadtaiyycariyngnsfpywgqgttvtvssggggggggsggggsdivmtqspdslavslgeratinckssqslfnsg |
| | | nqknyltwyqqkpgqppklliywastresgvpdrfsgsgsgtdftltisslqaedvavyycqnaysfpytfgggtkleikrTTTKPVLRTPSPVHPTGTSQP |
| | | QRPEDCRPRGSVKGTGLDFACDIYIWAPLAGICVALLLSLIITLICYHRSRKWIRKKFPHIFKQPFKKTTGAAQEE |
| | | DACSCRCPQEEEGGGGGYELSRSAETAANLQDPNQLYNELNLGRREEYDVLEKKRARDPEMGGKQQRRRNPQ |
| | | EGVYNALQKDKMAEAYSEIGTKGERRRGKGHDGLYQGLSTATKDTYDALHMQTLA |
| 23 | Amino acid | MAVTACQGLGFVVSLIGIAGIIAATCMDQWSTQDLYNNPVTAVFNYQGLWRSCVRESSGFTECRGYFTLLGLPA |
| | sequence of | MLQAVRALMIVGIVLGAIGLLVSIFALKCIRIGSMEDSAKANMTLTSGIMFIVSGLCAIAGVSVFANMLVTNFWM |
| | CLDN18.2 | STANMYTGMGGMVQTVQTRYTFGAALFVGWVAGGLTLIGGVMMCIACRGLAPEETNYKAVSYHASGHSVAY |
| | | KPGGFKASTGFGSNTKNKKIYDGGARTEDEVQSYPSKHDYV |
| 24 | Amino acid | lrpsrvhnseentmraltlkdilngtfsyktffpnwisgqeylhqsadnnivlynietgqsytilsnrtmksvnasnyglspdrqfvylesdysklwrysytatyyiydlsnge |
| | sequence of | fvrgnelprpiqylcwspvgsklayvyqnniylkqrpgdppfqitfngrenkifngipdwvyeeemlatkyalwwspngkflayaefndtdipviaysyygdeqyprti |
| | FAP | nipypkagaknpvvrifiidttypayvgpqevpvpamiassdyyfswltwvtdervclqwlkrvqnvsvlsicdfredwqtwdcpktqehieesrtgwaggffvstpvf |
| | | sydaisyykifsdkdgykhihyikdtvenaiqitsgkweainifrvtqdslfyssnefeeypgrrniyrisigsyppskkcvtchlrkercqyytasfsdyakyyalvcygpg |
| | | ipistlhdgrtdqeikileenkelenalkniqlpkeeikklevdeitlwykmilppqfdrskkyplliqvyggpcsqsvrsvfavnwisylaskegmvialvdgrgtafqgd |
| | | kllyavyrklgvyevedqitavrkfiemgfidekriaiwgwsyggyvsslalasgtglfkcgiavapvssweyyasvyterfmglptkddnlehyknstvmaraeyfrn |
| | | vdyllihgtaddnvhfqnsaqiakalvnaqvdfqamwysdqnhglsglstnhlythmthflkqcfslsd |
| 25 | Nucleotide | atggccgtgactgcctgtcagggcttggggttcgtggtttcactgattgggattggggcatcattgctgccacctgcatggaccagtggagcacccaagacttgtacaacaacc |
| | sequence of | ccgtaacagctgttttcaactaccaggggctgtggcgctcctgtgtccgagagagctctggcttcaccgagtgccggggctacttcaccctgctggggctgccagccatgctgc |
| | CLDN18.2 | aggcagtgcgagccctgatgatcgtaggcatcgtcctgggtgccattggcctcctggtatccatctttgccctgaaatgcatccgcattggcagcatggaggactctgccaaagc |
| | | caacatgacactgacctccgggatcatgttcattgtctcaggtctttgtgcaattgctggagtgtctgtgtttgccaacatgctggtgactaacttctggatgtccacagctaacatgt |
| | | acaccggcatgggtgggatggtgcagactgttcagaccaggtacacatttggtgcggctctgttcgtgggctgggtcgctggaggcctcacactaattgggggtgtgatgatgt |
| | | gcatcgcctgccggggcctggcaccagaagaaaccaactacaaagccgtttcttatcatgcctcaggccacagtgttgcctacaagcctggaggcttcaaggccagcactggc |
| | | tttgggtccaacaccaaaaacaagaagatatacgatggaggtgcccgcacagaggacgaggtacaatcttatccttccaagcacgactatgtgtaa |
| 26 | antiCLDN18. | SGYNWH |
| | 2-HCDR1 | |
| 27 | antiCLDN18. | yihytgstnynpalrs |
| | 2-HCDR2 | |
| 28 | antiCLDN18. | IYNGNSFPY |
| | 2-HCDR3 | |
| 29 | antiCLDN18. | KSSQSLFNSGNQKNYLT |
| | 2-LCDR1 | |
| 30 | antiCLDN18. | WASTRES |
| | 2-LCDR2 | |
| 31 | antiCLDN18. | QNAYSFPYT |
| | 2-LCDR3 | |
| 32 | Amino acid | Qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence-2 of | vtvssggggggggsggggsdivmtqspdslavslgeratinckssqslfnsgnqknyltwyqqkpgqppklliywastresgvpdrfsgsgsgtdftltisslqaedvav |
| | CLDN18.2- | yycqnaysfpytfgggtkleikrTTTPAPRPPTPAPTIASQPLSLRPEACRPAAGGAVHTRGLDFACDIYIWAPLAGTCGVLLL |
| | BBZ | SLVITLYCKRGRKKLLYIFKQPFMRPVQTTQEEDGCSCRFPEEEEGGCELRVKFSRSADAPAYKQGQNQLYNELN |
| | | LGRREEYDVLDKRRGRDPEMGGKPRRKNPQEGLYNELQKDKMAEAYSEIGMKGERRRGKGHDGLYQGLSTAT |
| | | KDTYDALHMQALPPR |
| 33 | Amino acid | Qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence of | vtvssggggggggsggggsdivmtqspdslavslgeratinckssqslfnsgnqknyltwyqqkpgqppklliywastresgvpdrfsgsgsgtdftltisslqaedvav |
| | CLDN18.2- | yycqnaysfpytfgggtkleikrTttpaprpptpaptiasqplslrpeacrpaaggavhtrgldfacdFwvlvvvggvlacysllvtvafiifwvRskrsrllhsdymnmtp |
| | 28Z | rrpgptrkhyqpyapprdfaayrsrvkfsrsadapayqqgqnqlynelnlgrreeydvldkrrgrdpemggkpqrrknpqeglynelqkdkmaeayseigmkgerrrg |
| | | kghdglyqglstatkdtydalhmqalppr |
| 34 | Amino acid | Qvqlqesgpglikpsqtlsltctvsggsissgynwhwirqppgkglewigyihytgstnynpalrsrvtisvdtsknqfslklssvtaadtaiyycariyngnsfpywgqgtt |
| | sequence of | vtvssggggggggsggggsdivmtqspdslavslgeratinckssqslfnsgnqknyltwyqqkpgqppklliywastresgvpdrfsgsgsgtdftltisslqaedvav |
| | CLDN18.2- | yycqnaysfpytfgggtkleikrTttpaprpptpaptiasqplslrpeacrpaaggavhtrgldfacdFwvlvvvggvlacysllvtvafiifwvRskrsrllhsdymnmtp |
| | 28BBZZ | rrpgptrkhyqpyapprdfaayrsKrgrkkllyifkqpfmrpvqttqeedgccrfpeeeeggcelrvkfsrsadapayqqgqnqlynelnlgrreeydvldkrrgrdpe |
| | | mggkpqrrknpqeglynelqkdkmaeayseigmkgerrrgkghdglyqglstatkdtydalhmqalppr |
| 35 | anti-FAP-LCD | RSSQSLLHPNGFNHLY |
| | R1 | |
| 36 | anti-FAP-LCD | VGGNRAS |
| | R2 | |
| 37 | anti-FAP-LCD | QQRNNKNRT |
| | R3 | |
| 38 | anti-FAP-HCD | SYAIS |
| | R1 | |
| 39 | anti-FAP-HCD | GIIPIFGTANYAQKFQG |
| | R2 | |
| 40 | anti-FAP-HCD | DAADRDY |
| | R3 | |
| 41 | Amino acid | QVQLQESGPGLIKPSQTLSLTCTVSGGSISSGYNWHWIRQPPGKGLEWIGYIHYTGSTNYNPALRSRVTISVDTSK |
| | sequence of | NQFSLKLSSVTAADTAIYYCARIYNGNSFPYWGQGTTVTVSSGGGGSGGGGSGGGGSDIVMTQSPDSLAVSLGE |
| | CLDN18.2-FA | RATINCKSSQSLFNSGNQKNYLTWYQQKPGQPPKLLIYWASTRESGVPDRFSGSGSGTDFTLTISSLQAEDVAVY |
| | P3-BBZ | YCQNAYSFPYTFGGGTKLEIKRGGGGSGGGGSGGGGSQVQLVQSGAEVKKPGSSVKVSCKASGGTFSSYAISW |
| | | VRQAPGQGLEWMGGIIPIFGTANYAQKFQGRVTITADKSTSTAYMELSSLRSEDTAVYYCARDAADRDYWGQG |
| | | TTVTVSSGGGGSGGGGSGGGGSDIVMTQSPLSLPVTPGEPASISCRSSQSLLHPNGFNHLYWYLQKPGQSPQLLI |
| | | YVGGNRASGVPDRFSGSGSGTDFTLKISRVEAEDVGVYYCQQRNNKNRTFGQGTKVEIKRTTTPAPRPPTPAPTI |
| | | ASQPLSLRPEACRPAAGGAVHTRGLDFACDIYIWAPLAGTCGVLLLSLVITLYCKRGRKKLLYIFKQPFMRPVQT |
| | | TQEEDGCSCRFPEEEEGGCELRVKFSRSADAPAYQQGQNQLYNELNLGRREEYDVLDKRRGRDPEMGGKPQRR |
| | | KNPQEGLYNELQKDKMAEAYSEIGMKGERRRGKGHDGLYQGLSTATKDTYDALHMQALPPR |
| 42 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | FAP-CLDN18. | EPASISCRSSQSLLHPNGFNHLYWYLQKPGQSPQLLIYVGGNRASGVPDRFSGSGSGTDFTLKISRVEAEDVGVY |
| | 2-BBZ | YCQQRNNKNRTFGQGTKVEIKRGGGGSGGGGSGGGGSQVQLQESGPGLIKPSQTLSLTCTVSGGSISSGYNWH |
| | | WIRQPPGKGLEWIGYIHYTGSTNYNPALRSRVTISVDTSKNQFSLKLSSVTAADTAIYYCARIYNGNSFPYWGQG |
| | | TTVTVSSGGGGSGGGGSGGGGSDIVMTQSPDSLAVSLGERATINCKSSQSLFNSGNQKNYLTWYQQKPGQPPKL |
| | | LIYWASTRESGVPDRFSGSGSGTDFTLTISSLQAEDVAVYYCQNAYSFPYTFGGGTKLEIKRTTTPAPRPPTPAPTI |
| | | ASQPLSLRPEACRPAAGGAVHTRGLDFACDIYIWAPLAGTCGVLLLSLVITLYCKRGRKKLLYIFKQPFMRPVQT |
| | | TQEEDGCSCRFPEEEEGGCELRVKFSRSADAPAYQQGQNQLYNELNLGRREEYDVLDKRRGRDPEMGGKPQRR |
| | | KNPQEGLYNELQKDKMAEAYSEIGMKGERRRGKGHDGLYQGLSTATKDTYDALHMQALPPR |
| 43 | Amino acid | QVQLVQSGAEVKKPGSSVKVSCKASGGTFSSYAISWVRQAPGQGLEWMGGIIPIFGTANYAQKFQGRVTITADK |
| | sequence-1 of | STSTAYMELSSLRSEDTAVYYCARDAADRDYWGQGTTVTVSSGGGGSGGGGSGGGGSDIVMTQSPLSLPVTPG |
| | FAP-BBZ | EPASISCRSSQSLLHPNGFNHLYWYLQKPGQSPQLLIYVGGNRASGVPDRFSGSGSGTDFTLKISRVEAEDVGVY |
| | | YCQQRNNKNRTFGQGTKVEIKRTTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQKDKMAEAYSEIGMKGERRRGKGHDGL |
| | | YQGLSTATKDTYDALHMQALPPR |
| 44 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | FAP-28Z | EPASISCRSSQSLLHPNGFNHLYWYLQKPGQSPQLLIYVGGNRASGVPDRFSGSGSGTDFTLKISRVEAEDVGVY |
| | | YCQQRNNKNRTFGQGTKVEIKRTttpaprpptpaptiasqplslrpeacrpaaggavhtrgldfacdFwvlvvvggvlacysllvtvafiifwvRskrsrl |
| | | lhsdymnmtprrpgptrkhyqpyapprdfaayrsrvkfsrsadapayqqgqnqlynelnlgrreeydvldkrrgrdpemggkpqrrknpqeglynelqkdkmaeay |
| | | seigmkgerrrgkghdglyqglstatkdtydalhmqalppr |
| 45 | Amino acid | QVQLVQSGAEVKKPGSSVKVSCKASGGTFSSYAISWVRQAPGQGLEWMGGIIPIFGTANYAQKFQGRVTITADK |
| | sequence of | STSTAYMELSSLRSEDTAVYYCARDAADRDYWGQGTTVTVSSGGGGSGGGGSGGGGSDIVMTQSPLSLPVTPG |
| | FAP-28BBZZ | EPASISCRSSQSLLHPNGFNHLYWYLQKPGQSPQLLIYVGGNRASGVPDRFSGSGSGTDFTLKISRVEAEDVGVY |
| | | YCQQRNNKNRTFGQGTKVEIKRTttpaprpptpaptiasqplslrpeacrpaaggavhtrgldfacdFwvlvvvggvlacysllvtvafiifwvRskrsrl |
| | | lhsdymnmtprrpgptrkhyqpyapprdfaayrsKrgrkkllyifkqpfmrpvqttqeedgccrfpeeeeggcelrvkfsrsadapayqqgqnqlynelnlgrreeyd |
| | | vldkrrgrdpemggkpqrrknpqeglynelqkdkmaeayseigmkgerrrgkghdglyqglstatkdtydalhmqalppr |
| 46 | Amino acid | QVQLVQSGAEVKKPGSSVKVSCKASGGTFSSYAISWVRQAPGQGLEWMGGIIPIFGTANYAQKFQGRVTITADK |
| | sequence-2 of | STSTAYMELSSLRSEDTAVYYCARDAADRDYWGQGTTVTVSSGGGGSGGGGSGGGGSDIVMTQSPLSLPVTPG |
| | FAP-BBZ | EPASISCRSSQSLLHPNGFNHLYWYLQKPGQSPQLLIYVGGNRASGVPDRFSGSGSGTDFTLKISRVEAEDVGVY |
| | | YCQQRNNKNRTFGQGTKVEIKRTttpaprpptpaptiasqplslrpeacrpaaggavhtrgldfacdIyiwaplagtcgvlllslvitlycKrgrkkllyifk |
| | | qpfmrpvqttqeedgccrfpeeeeggcelrvkfsrsadapayqqgqnqlynelnlgrreeydvldkrrgrdpemggkpqrrknpqeglynelqkdkmaeayseigm |
| | | kgerrrgkghdglyqglstatkdtydalhmqalppr |

1. A multifunctional immune effector cell, wherein the immune effector cell expresses a protein specifically recognizing FAP and a protein specifically recognizing a tumor-associated antigen.

2. The immune effector cell according to claim 1, wherein the tumor-associated antigen is a solid tumor-associated antigen;
preferably, the solid tumor-associated antigen is an antigen associated with breast cancer, liver cancer, gastric cancer, colorectal cancer, ovarian cancer, lung cancer, or pancreatic cancer;
more preferably, the solid tumor is pancreatic cancer; or the solid tumor-associated antigen is Claudin 18.2.

3. The immune effector cell according to claim 1, wherein the cell is selected from the group consisting of: T cell, NK cell, NKT cell, macrophage, CIK cell, and stem cell-derived immune effector cell;
preferably, the cell is T cell.

4. The immune effector cell according to claim 1, wherein the protein specifically recognizing FAP and the protein specifically recognizing a tumor-associated antigen are expressed into a fusion protein by fused expression;
preferably, the fusion protein is connected with a transmembrane domain and an intracellular signal domain to form a chimeric receptor,
more preferably, the chimeric receptor comprises a protein specifically recognizing FAP, a protein specifically recognizing a tumor-associated antigen, a transmembrane domain and an intracellular signal domain which are connected in sequence; alternatively, the chimeric receptor comprises a protein specifically recognizing a tumor-associated antigen, a protein specifically recognizing FAP, a transmembrane domain and an intracellular signal domain which are connected in sequence.

5. (canceled)

6. The immune effector cell according to claim 1,
wherein the protein specifically recognizing FAP comprises an antibody targeting FAP or a ligand of FAP;
preferably, the antibody targeting FAP is a single chain antibody or a single domain antibody;
more preferably, the single chain antibody has LCDR1, LCDR2 and LCDR3 represented by SEQ ID NOs: 35, 36 and 37, and HCDR1, HCDR2 and HCDR3 represented by SEQ ID NOs: 38, 39 and 40;
more preferably, the single chain antibody has the amino acid sequence represented by SEQ ID NO: 2; or
wherein the protein specifically recognizing a tumor-associated antigen is an antibody specifically recognizing a tumor antigen or a ligand of a tumor antigen;
preferably, the antibody specifically recognizing a tumor antigen is a single chain antibody or a single domain antibody;
more preferably, the single chain antibody has LCDR1, LCDR2 and LCDR3 represented by SEQ ID NOs: 29, 30 and 31, and HCDR1, HCDR2 and HCDR3 represented by SEQ ID NOs: 26, 27 and 28;
more preferably, the single chain antibody has the amino acid sequence represented by SEQ ID NO: 4.

7. (canceled)

8. The immune effector cell according to claim 4, wherein the chimeric receptor is selected from the group consisting of: chimeric antigen receptor (CAR), chimeric T cell receptor, or T cell antigen coupler (TAC).

9. The immune effector cell according to claim 4, wherein the protein specifically recognizing FAP and the protein specifically recognizing a tumor-associated antigen are connected through a connecting peptide, preferably the protein specifically recognizing a tumor-associated antigen is located upstream of the protein specifically recognizing FAP.

10. The immune effector cell according to claim 4 or 5, wherein the intracellular signal domain is selected from the intracellular signal domain sequences of CD3ε FcεRIγ, CD27, CD28, CD137 and CD134, or a combination thereof:
preferably, the chimeric receptor comprises an extracellular binding domain, a transmembrane domain and an intracellular signal domain which are connected in the following order:
a fusion protein, a transmembrane domain of CD8, and an intracellular domain of CD3ζ;
a fusion protein, a transmembrane domain of CD8, an intracellular signal domain of CD137, and an intracellular domain of CD3ζ;
a fusion protein, a transmembrane domain of CD28, an intracellular signal domain of CD28, and an intracellular domain of CD3ζ; or
a fusion protein, a transmembrane domain of CD28, an intracellular signal domain of CD28, an intracellular signal domain of CD137, and intracellular domain of CD3ζ.

11. (canceled)

12. The immune effector cell according to claim 1, wherein the protein specifically recognizing FAP and the protein specifically recognizing a tumor-associated antigen are expressed separately, preferably, the protein specifically recognizing FAP is a chimeric receptor which comprises an antibody targeting FAP or a ligand of FAP, a transmembrane domain, and an intracellular signal domain; or the protein specifically recognizing a tumor-associated antigen is a chimeric receptor which comprises an antibody targeted-binding a tumor antigen or a ligand of a tumor antigen, a transmembrane domain, and an intracellular signal domain.

13. (canceled)

14. (canceled)

15. The immune effector cell according to claim 12, wherein the protein specifically recognizing FAP is a chimeric receptor A which comprises an antibody targeting FAP or a ligand of FAP, a transmembrane domain and an intracellular signal domain; and the protein specifically recognizing a tumor-associated antigen is a chimeric receptor B which comprises an antibody targeted-binding a tumor antigen or a ligand of a tumor antigen, a transmembrane domain, and an intracellular signal domain;
preferably the chimeric receptor A and the chimeric receptor B have the same intracellular signal domain or different intracellular signal domains;
more preferably the intracellular signal domain is selected from the intracellular signal domain sequences of CD3ζ, FcεRIγ, CD27, CD28, CD137 and CD134, or a combination thereof;
preferably, the chimeric receptor A has the amino acid sequence represented by SEQ ID NO: 43, 44, 45 or 46; or the chimeric receptor B has the amino acid sequence represented by SEQ ID NO: 16, 32, 33 or 34.

16. (canceled)

17. (canceled)

18. The immune effector cell according to claim 10, wherein the chimeric receptor has the amino acid sequence represented by SEQ ID NO: 41, SEQ ID NO: 20, SEQ ID NO: 22 or SEQ ID NO: 42;
preferably, the chimeric receptor has the amino acid sequence represented by SEQ ID NO: 41 or 42.

19. A fusion protein, wherein it comprises a protein targeting FAP, and a protein specifically recognizing a tumor-associated antigen,
preferably, the tumor-associated antigen is a solid tumor-associated antigen;
preferably, the solid tumor-associated antigen is an antigen associated with breast cancer, liver cancer, gastric cancer, colorectal cancer, ovarian cancer, lung cancer, or pancreatic cancer;
more preferably, the solid tumor-associated antigen is Claudin 18.2.

20. (canceled)

21. The fusion protein according to claim 19, wherein the fusion protein is connected with a transmembrane domain and an intracellular signal domain to form a chimeric receptor;
preferably the chimeric receptor comprises a protein specifically recognizing FAP, a protein specifically recognizing a tumor-associated antigen, a transmembrane domain and an intracellular signal domain which are connected in sequence; alternatively, the chimeric receptor comprises a protein specifically recognizing a tumor-associated antigen, a protein specifically recognizing FAP, a transmembrane domain and an intracellular signal domain which are connected in sequence.

22. (canceled)

23. The fusion protein according to claim 19, wherein the protein specifically recognizing FAP comprises an antibody targeting FAP or a ligand of FAP;
preferably, the antibody targeting FAP is a single chain antibody or a single domain antibody;
more preferably, the single chain antibody has the amino acid sequence represented by SEQ ID NO: 2 or;
wherein the protein specifically recognizing a tumor-associated antigen is an antibody specifically recognizing a tumor antigen or a ligand of a tumor antigen;
preferably, the antibody specifically recognizing a tumor antigen is a single chain antibody or a single domain antibody;
more preferably, the single chain antibody has the amino acid sequence represented by SEQ ID NO: 4.

24. (canceled)

25. The fusion protein according to claim 21, wherein the chimeric receptor is selected from the group consisting of: chimeric antigen receptor (CAR), chimeric T cell receptor, and T cell antigen coupler (TAC).

26. The fusion protein according to claim 19, wherein the protein specifically recognizing FAP and the protein specifically recognizing a tumor-associated antigen are connected through a connecting peptide, preferably the protein specifically recognizing a tumor-associated antigen is located upstream of the protein specifically recognizing FAP.

27. The fusion protein according to claim 22, wherein the intracellular signal domain is selected from the intracellular signal domain sequences of CD3ζ, FcεRIγ, CD27, CD28, CD137 and CD134, or a combination thereof,
preferably, the chimeric receptor comprises an extracellular binding domain, a transmembrane domain and an intracellular signal domain which are connected in the following order:
a fusion protein, a transmembrane domain of CD8, and an intracellular domain of CD3ζ;
a fusion protein, a transmembrane domain of CD8, an intracellular signal domain of CD137, and an intracellular domain of CD3ζ;
a fusion protein, a transmembrane domain of CD28, an intracellular signal domain of CD28, and an intracellular domain of CD3ζ; or
a fusion protein, a transmembrane domain of CD28, an intracellular signal domain of CD28, an intracellular signal domain of CD137, and intracellular domain of CD3ζ.

28. (canceled)

29. A nucleic acid encoding the fusion protein according to claim 19.

30. (canceled)

31. (canceled)

32. A pharmaceutical composition, wherein it comprises:
the immune effector cell according to claim 1, and/or the fusion protein according to claim 19; and
a pharmaceutically acceptable carrier.

33. (canceled)

34. A method for treating a tumor, wherein the immune effector cells according to claim 1 are administered to an individual suffering from a tumor, preferably the lymphocytes of the individual are eliminated before administration of the immune effector cells,
wherein preferably, the tumor is a tumor rich in a large number of CAFs cells in the tumor microenvironment;
preferably, the tumor is breast cancer, liver cancer, gastric cancer, lung cancer, or pancreatic cancer;
more preferably, the tumor is pancreatic cancer.

35. (canceled)


SEQUENCE LISTING (46 sequences)

SEQ ID NO: 1 - DNA, 732 bp, Artificial Sequence
  Description of Artificial Sequence artificially synthesized sequence
caggtgcaattggtgcagtctggggctgaggtgaagaagcctgggtcctcggtgaaggtctcctgcaaggcctccggaggcacattcagcagctacgctataagctgggtgcgacaggcccctggacaagggctcgagtggatgggagggatcatccctatctttggtacagcaaactacgcacagaagttccagggcagggtcaccattactgcagacaaatccacgagcacagcctacatggagctgagcagcctgagatctgaggacaccgccgtgtattactgtgcgagagatgctgctgatagggactactggggccaagggaccaccgtgaccgtctcctcaggtggaggcggttcaggcggaggtggttctggcggtggcggatcggatattgttatgactcaatctccactgtctctgccggtgactccaggcgaaccggcgagcatttcttgccgttccagccagtctctgcttcaccccaacggcttcaaccatctctattggtacctgcaaaaaccgggtcagagccctcagctgctgatctacgtggggggtaaccgcgcttccggtgtaccggaccgtttcagcggctctggatccggcaccgatttcacgttgaaaatcagccgtgttgaagcagaagacgtgggcgtttattactgtcagcagcgtaataataagaatcgtacttttggtcaaggcaccaaggtcgaaattaaacgt

SEQ ID NO: 2 - protein, 244 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKR

SEQ ID NO: 3 - DNA, 741 bp, Artificial Sequence
  Description of Artificial Sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 4 - protein, 247 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 5 - DNA, 81 bp, Artificial Sequence
  Description of Artificial Sequence artificially synthesized sequence
atggcctcaccgttgacccgctttctgtcgctgaacctgctgctgctgggtgagtcgattatcctggggagtggagaagct

SEQ ID NO: 6 - protein, 27 aa, Artificial Sequence
  Description of Artificial Sequence artificially synthesized sequence
MASPLTRFLSLNLLLLGESIILGSGEA

SEQ ID NO: 7 - DNA, 216 bp, Artificial Sequence
  Description of Artificial Sequence artificially synthesized sequence
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccga

SEQ ID NO: 8 - protein, 72 aa, Artificial Sequence
  Description of Artificial Sequence artificially synthesized sequence
TTTKPVLRTPSPVHPTGTSQPQRPEDCRPRGSVKGTGLDFACDIYIWAPLAGICVALLLSLIITLICYHRSR

SEQ ID NO: 9 - DNA, 135 bp, Artificial Sequence
  Description of Artificial Sequence artificially synthesized sequence
aaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 10 - protein, 45 aa, Artificial Sequence
  Description of Artificial Sequence artificially synthesized sequence
KWIRKKFPHIFKQPFKKTTGAAQEEDACSCRCPQEEEGGGGGYEL

SEQ ID NO: 11 - DNA, 321 bp, Artificial Sequence
  Description of Artificial Sequence artificially synthesized sequence
agcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 12 - protein, 107 aa, Artificial Sequence
  Description of Artificial Sequence artificially synthesized sequence
SRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 13 - DNA, 45 bp, Artificial Sequence
  Description of Artificial Sequence artificially synthesized sequence
ggtggaggcggttcaggcggaggtggttctggcggtggcggatcg

SEQ ID NO: 14 - protein, 15 aa, Artificial Sequence
  Description of Artificial Sequence artificially synthesized sequence
GGGGSGGGGSGGGGS

SEQ ID NO: 15 - DNA, 1413 bp, Artificial Sequence
  Description of Artificial Sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 16 - protein, 471 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - DNA, 1404 bp, Artificial Sequence
  Description of Artificial Sequence artificially synthesized sequence
caggtgcaattggtgcagtctggggctgaggtgaagaagcctgggtcctcggtgaaggtctcctgcaaggcctccggaggcacattcagcagctacgctataagctgggtgcgacaggcccctggacaagggctcgagtggatgggagggatcatccctatctttggtacagcaaactacgcacagaagttccagggcagggtcaccattactgcagacaaatccacgagcacagcctacatggagctgagcagcctgagatctgaggacaccgccgtgtattactgtgcgagagatgctgctgatagggactactggggccaagggaccaccgtgaccgtctcctcaggtggaggcggttcaggcggaggtggttctggcggtggcggatcggatattgttatgactcaatctccactgtctctgccggtgactccaggcgaaccggcgagcatttcttgccgttccagccagtctctgcttcaccccaacggcttcaaccatctctattggtacctgcaaaaaccgggtcagagccctcagctgctgatctacgtggggggtaaccgcgcttccggtgtaccggaccgtttcagcggctctggatccggcaccgatttcacgttgaaaatcagccgtgttgaagcagaagacgtgggcgtttattactgtcagcagcgtaataataagaatcgtacttttggtcaaggcaccaaggtcgaaattaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 18 - protein, 468 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 19 - DNA, 2190 bp, Artificial Sequence
  Description of Artificial Sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggggtggaggcggttcaggcggaggtggttctggcggtggcggatcgcaggtgcaattggtgcagtctggggctgaggtgaagaagcctgggtcctcggtgaaggtctcctgcaaggcctccggaggcacattcagcagctacgctataagctgggtgcgacaggcccctggacaagggctcgagtggatgggagggatcatccctatctttggtacagcaaactacgcacagaagttccagggcagggtcaccattactgcagacaaatccacgagcacagcctacatggagctgagcagcctgagatctgaggacaccgccgtgtattactgtgcgagagatgctgctgatagggactactggggccaagggaccaccgtgaccgtctcctcaggtggaggcggttcaggcggaggtggttctggcggtggcggatcggatattgttatgactcaatctccactgtctctgccggtgactccaggcgaaccggcgagcatttcttgccgttccagccagtctctgcttcaccccaacggcttcaaccatctctattggtacctgcaaaaaccgggtcagagccctcagctgctgatctacgtggggggtaaccgcgcttccggtgtaccggaccgtttcagcggctctggatccggcaccgatttcacgttgaaaatcagccgtgttgaagcagaagacgtgggcgtttattactgtcagcagcgtaataataagaatcgtacttttggtcaaggcaccaaggtcgaaattaaacgtactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 20 - protein, 730 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRGGGGSGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 21 - DNA, 2190 bp, Artificial Sequence
  Description of Artificial Sequence artificially synthesized sequence
caggtgcaattggtgcagtctggggctgaggtgaagaagcctgggtcctcggtgaaggtctcctgcaaggcctccggaggcacattcagcagctacgctataagctgggtgcgacaggcccctggacaagggctcgagtggatgggagggatcatccctatctttggtacagcaaactacgcacagaagttccagggcagggtcaccattactgcagacaaatccacgagcacagcctacatggagctgagcagcctgagatctgaggacaccgccgtgtattactgtgcgagagatgctgctgatagggactactggggccaagggaccaccgtgaccgtctcctcaggtggaggcggttcaggcggaggtggttctggcggtggcggatcggatattgttatgactcaatctccactgtctctgccggtgactccaggcgaaccggcgagcatttcttgccgttccagccagtctctgcttcaccccaacggcttcaaccatctctattggtacctgcaaaaaccgggtcagagccctcagctgctgatctacgtggggggtaaccgcgcttccggtgtaccggaccgtttcagcggctctggatccggcaccgatttcacgttgaaaatcagccgtgttgaagcagaagacgtgggcgtttattactgtcagcagcgtaataataagaatcgtacttttggtcaaggcaccaaggtcgaaattaaacgtggtggaggcggttcaggcggaggtggttctggcggtggcggatcgcaggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 22 - protein, 730 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRGGGGSGGGGSGGGGSQVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 23 - protein, 261 aa, Artificial Sequence
  Description of Artificial Sequence artificially synthesized sequence
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 24 - protein, 735 aa, Artificial Sequence
  Description of Artificial Sequence artificially synthesized sequence
LRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 25 - DNA, 786 bp, Artificial Sequence
  Description of Artificial Sequence artificially synthesized sequence
atggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaa

SEQ ID NO: 26 - protein, 6 aa, Artificial Sequence
  Description of Artificial Sequence artificially synthesized sequence
SGYNWH

SEQ ID NO: 27 - protein, 16 aa, Artificial Sequence
  Description of Artificial Sequence artificially synthesized sequence
YIHYTGSTNYNPALRS

SEQ ID NO: 28 - protein, 9 aa, Artificial Sequence
  Description of Artificial Sequence artificially synthesized sequence
IYNGNSFPY

SEQ ID NO: 29 - protein, 17 aa, Artificial Sequence
  Description of Artificial Sequence artificially synthesized sequence
KSSQSLFNSGNQKNYLT

SEQ ID NO: 30 - protein, 7 aa, Artificial Sequence
  Description of Artificial Sequence artificially synthesized sequence
WASTRES

SEQ ID NO: 31 - protein, 9 aa, Artificial Sequence
  Description of Artificial Sequence artificially synthesized sequence
QNAYSFPYT

SEQ ID NO: 32 - protein, 470 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 473 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 34 - protein, 515 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 16 aa, Artificial Sequence
  Description of Artificial Sequence artificially synthesized sequence
RSSQSLLHPNGFNHLY

SEQ ID NO: 36 - protein, 7 aa, Artificial Sequence
  Description of Artificial Sequence artificially synthesized sequence
VGGNRAS

SEQ ID NO: 37 - protein, 9 aa, Artificial Sequence
  Description of Artificial Sequence artificially synthesized sequence
QQRNNKNRT

SEQ ID NO: 38 - protein, 5 aa, Artificial Sequence
  Description of Artificial Sequence artificially synthesized sequence
SYAIS

SEQ ID NO: 39 - protein, 17 aa, Artificial Sequence
  Description of Artificial Sequence artificially synthesized sequence
GIIPIFGTANYAQKFQG

SEQ ID NO: 40 - protein, 7 aa, Artificial Sequence
  Description of Artificial Sequence artificially synthesized sequence
DAADRDY

SEQ ID NO: 41 - protein, 730 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRGGGGSGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 730 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRGGGGSGGGGSGGGGSQVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 467 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 470 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - protein, 512 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 468 aa, Artificial Sequence
  Description of Artificial Sequence artificially synthesized sequence
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDAADRDYWGQGTTVTVSSGGGGSGGGGSGGGGSDIVMTQSPLSLPVTPGEPASISCRSSQSLLHPNGFNHLYWYLQKPGQSPQLLIYVGGNRASGVPDRFSGSGSGTDFTLKISRVEAEDVGVYYCQQRNNKNRTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR